(19) United States
(12) Patent Application Publication
(10) Pub. No.: US 2020/0306476 A1
(43) Pub. Date: Oct. 1, 2020

(54) Hydro-gravitational method and device for lung refurbishment

(71) Applicants: Victor Popa-Simil, Los Alamos, NM (US); Andrei Popa-Simil, Los Alamos, NM (US)

(72) Inventors: Victor Popa-Simil, Los Alamos, NM (US); Andrei Popa-Simil, Los Alamos, NM (US)

(73) Assignee: LAVM LLC, Los Alamos, NM (US)

(21) Appl. No.: 16/847,926

(22) Filed: Apr. 14, 2020

(51) Int. Cl. A61M 16/04; A61M 16/20; A61M 16/10; A61M 16/00; A61L 2/08; G16H 20/40

(52) U.S. Cl. CPC A61M 16/0481; A61M 2205/3306; A61M 16/201; A61M 16/10; A61M 16/0006; A61L 2/08; G16H 20/40; A61M 2016/003; A61M 2205/583; A61M 2205/3368; A61M 2205/3324; A61M 2205/3396; A61M 2205/3331; A61M 2205/82; A61M 2205/35; A61M 2210/1039; A61L 2202/22; A61M 16/044

(57) ABSTRACT

Many pneumonia diseases and lung malfunctions can be quickly repaired using an improved lung lavage technique where the patient is rotated in specific 3D orientations to increase the efficiency of the lavage procedure. The process involves filling and emptying the lungs with fluid and rotating the patient makes this process “natural” and effective. Supplementary, a hydro-pneumatic system facilitates the operations with the patient sustained in various positions such as being immersed in water and having various control mechanisms such as variable pressures, temperatures, and performing assisted breathing. Additionally, immersed devices are implanted that “shake-up of alveolar wall” and other devices perform ultrasound imaging with a 0.1 mm resolution, a resolution in competition with stereoscopic X-ray. The bio-medical data acquisition system allows physicians to completely assess patient status in real time and guide the treatment to ensure optimum patient care, under quality assurance procedures.

Many pneumonia diseases and lung malfunctions can be quickly repaired using an improved lung lavage technique where the patient is rotated in specific 3D orientations to increase the efficiency of the lavage procedure. The process involves filling and emptying the lungs with fluid and rotating the patient makes this process “natural” and effective. Supplementary, a hydro-pneumatic system facilitates the operations with the patient sustained in various positions such as being immersed in water and having various control mechanisms such as variable pressures, temperatures, and performing assisted breathing. Additionally, immersed devices are implanted that “shake-up of alveolar wall” and other devices perform ultrasound imaging with a 0.1 mm resolution, a resolution in competition with stereoscopic X-ray. The bio-medical data acquisition system allows physicians to completely assess patient status in real time and guide the treatment to ensure optimum patient care, under quality assurance procedures.

### STATEMENT RECARDINC FEDERALLY SPONSORED R&D

This invention was made with NO Government support.

### NAMES OF PARTIES TOA JOINT RESEARCH AGREEMENT

This work was part of research of the mentioned inventors.

### CROSS REFERENCE TO RELATED APPLICATIONS

This Application claims no priority.

### BACKGROUND OF THE INVENTION

### I. Field of the Invention

The present invention relates to a method and device to wash the lungs of patients that are clogged from various conditions and substances such as pneumonia or dust particulates. The procedures according to invention clean and treats the lungs' interior as well as improving their overall performance and recovering from age related wear.

The technology Is presently known under the name of lung lavage, but this is performed with patient on their back, making it difficult to completely drain the lungs. However, the patient may be set in the cockpit of a flight simulator as that in the U.S. Pat. No. 9,984,586. Placing the patient in that environment allows for the patient to be turned and rotated to completely draining the lungs like a bottle. The drained fluid and softened mucus would be collected and then the patient would again be flipped to allow for the filling of the lungs with the next solution. The process of rotating and flipping the patient between filling and draining stages may be repeated until the operation that includes, primary washing and softening of mucus and other residues, cleaning and evacuating mucus softened by the primary lavage solution, rinse, refill with healing solution, and final wash with alveoli performance enhancing solution, is completed.

The invention seeks to improve the current method by improving the quality of alveolar wall washing and diversification of substances for various types of contaminants that may be found inside; ranging from dust to mucus and other liquids.

The method is aiming to extend the treatment area and be a common method to reset the respiratory function after a large pallet of potential incidents affects lung functionality, the incidents of which include but are not limited to aerosol particulates, dust, smoke, gas, bacteria, and liquid effluents.

The system consists of a variety of devices that allow for treatment with the patient under anesthesia but not connected to any life support device because only a portion of the lung undergoes the rinsing procedure at a time. At the same time, approximately ¾ of the lung will be supplied with oxygen enriched air.

This method consists of a set of procedures to increase washing and liquid agitation inside saturated alveoli and to acquire data on the) washing process and lungs state of functionality to predict with anticipation the next necessary actions.

### 2. DESCRIPTION OF THE PRIOR ART

WebMD.com states; “Lung diseases are some of the most common medical conditions in the world. Tens of millions of people suffer from lung disease in the U.S. Smoking, infections, and genetics are responsible for most lung diseases. The lungs are part of a complex apparatus, expanding and relaxing thousands of times each day to bring in oxygen and expel carbon dioxide. Lung disease can result from problems in any part of this system.

There are some lung diseases that are affecting the airways, as trachea (windpipe) that branches into tubes called bronchi, which in turn branch to become progressively smaller tubes throughout the lungs. Diseases that affect the airways include:

Asthma: The airways are persistently inflamed, and may occasionally spasm, causing wheezing and shortness of breath. Allergies, infections, or pollution can trigger asthma's symptoms.

Chronic obstructive pulmonary disease (COPD): Lung conditions defined by an inability to exhale normally, which causes difficulty breathing.

Chronic bronchitis: A form of COPD characterized by a chronic productive cough,

Emphysema: Lung damage allows air to be trapped in the lungs in this form of COPD. Difficulty blowing air out is its hallmark.

Acute bronchitis: A sudden infection of the airways, usually by a virus.

Cystic fibrosis: A genetic condition causing poor clearance of mucus from the bronchi. The accumulated mucus results in repeated lung infections.

Other Lung Diseases are affecting the air sacs (Alveoli) that airways eventually branch into tiny tubes (bronchioles) that dead-end into clusters of air sacs called alveoli. These air sacs make up most of the lung tissue.

Lung diseases affecting the alveoli include:
Pneumonia: An infection of the alveoli, usually by bacteria.
Tuberculosis: A slowly progressive pneumonia caused by the bacteria Mycobacterium tuberculosis.
Emphysema results from damage to the fragile connections between alveoli. Smoking is the usual cause. (Emphysema also limits airflow, affecting the airways as well.)
Pulmonary edema: Fluid leaks out of the small blood vessels of the lung into the air sacs and the surrounding area. One form is caused by heart failure and back pressure in the lungs' blood vessels; in another form, direct injury to the lung causes the leak of fluid.
Lung cancer has many forms, and may develop in any part of the lungs, Most often this is in the main part of the lung, in or near the air sacs. The type, location, and spread of lung cancer determine the treatment options.
Acute respiratory distress syndrome (ARDS): Severe, sudden injury to the lungs caused by a serious illness. Life support with mechanical ventilation is usually needed to survive until the lungs recover.
Pneumoconiosis; A category of conditions caused by the inhalation of a substance that injures the lungs. Examples include black lung disease from inhaled coal dust and asbestosis from inhaled asbestos dust.”

Lung lavage is a relatively new process where not so many clinics are performing it although it has the potential to be successfully applied against any intrusion that attacks the lung's surface. Developing the most appropriate chemicals and drugs to wash, treat, and refurbish the surface will be an important factor of success.

To see potential issues inside lungs, physicians can use a bronchoscope (a thin, tube-like instrument with a light and a lens for viewing) that is inserted through the nose or mouth and down into the lungs. Then a mild salt solution is washed over the surface of the airways to collect cells, which are then looked at under a microscope. Bronchial washing is then used to find infections.

Another way to find how much a lung is affected is to perform an X ray chest radiography, or the measure the efficiency of oxygen to carbon dioxide conversion, usually for normal lungs it is about 4-5%, while a man produces 2.3 lb CO₂/day, and correlates with the respiratory volume.

The main apparatus at work that makes the respiratory function is alveoli, which according to healthline.com: “ . . . are tiny air sacs in your lungs that take up the oxygen you breathe in and keep your body going. Although they're microscopic, alveoli are the workhorses of your respiratory system.” A normal person has about 480 million alveoli, located at the end of the bronchial tubes. When inspiration occurs, the alveoli expand to take in oxygen, and then shrink to expel carbon dioxide during expiration.

Healthline also states: “There are three overall processes involved in your breathing:
moving air in and out of your lungs (ventilation)
oxygen-carbon dioxide exchange (diffusion)
pumping blood through your lungs (perfusion)

As it moves through blood vessels (capillaries) in the alveoli walls, your blood takes the oxygen from the alveoli and gives off carbon dioxide to the alveoli. These tiny alveoli structures taken all together form a very large surface area to do the work of breathing, cover a surface that measures more than 1,076.4 square feet (100 square meters). This large surface area is necessary to process the huge amounts of air involved in breathing and getting oxygen to lungs that take in about 1.3 to 2.1 gallons (5 to 8 liters) of air per minute, and when at rest, the alveoli send 10.1 ounces (0.3 liters) of oxygen to your blood per minute. To push the air in and out, diaphragm and other muscles help create pressure inside chest. When breathe in, muscles create a negative pressure—less than the atmospheric pressure that helps suck air in. When breathe out, the lungs recoil and return to their normal size.”

Healthline also states: “Lungs are two well-branched tree limbs, one on each side of your chest. The right lung has three sections (lobes), and the left lung has two sections (above the heart). The larger branches in each lobe are called bronchi. The bronchi divide into smaller branches called bronchioles. And at the end of each bronchiole is a small duct (alveolar duct) that connects to a cluster of thousands of microscopic bubble-like structures, the alveoli that are organized into bunches, each bunch grouped is what's called the alveolar sac. The alveoli touch each other, like grapes in a tight bunch. The number of alveoli and alveolar sacs are what give your lungs a spongy consistency. Each alveolus (singular of alveoli) is about 0.2 millimeters in diameter (about 0.008 inches). Each alveolus is cup-shaped with very thin walls. It's surrounded by networks of blood vessels called capillaries that also have thin walls.

The oxygen one breathes in diffuses through the alveoli and the capillaries into the blood. The carbon dioxide one breathes out is diffused from the capillaries to the alveoli, up the bronchial tree and out through mouth. The alveoli are just one cell in thickness, which allows the gas exchange of respiration to take place rapidly. The wall of an alveolus and the wall of a capillary are each about 0.00004 inches about 1 μm. There are two types of cells:

Type 1 alveoli cells cover 95 percent of the alveolar surface and constitute the air-blood barrier.

Type 2 alveoli cells are smaller and responsible for producing the surfactant that coats the inside surface of the alveolus and helps reduce surface tension. The surfactant helps keep the shape of each alveolus when you breathe in and out. They can also turn into stem cells, if necessary to repair injured alveoli, and become new alveoli cells.

This seemingly perfect machine for breathing can break down or become less efficient because of: disease, normal aging, smoking and air pollution.

Smoking tobacco injures lungs and leads to lung diseases like chronic obstructive pulmonary disease (COPD), emphysema, and chronic bronchitis, irritates bronchioles and alveoli and damages the lining of lungs. Tobacco damage is cumulative. Years of exposure to cigarette smoke can scar lung tissue so that lungs can't efficiently process oxygen and carbon dioxide. The damage from smoking isn't reversible.

Indoor pollution from secondhand smoke, mold, dust, household chemicals, radon, or asbestos can damage lungs and worsen existing lung disease, while outdoor pollution, such as car or industrial emissions, is also harmful. Chronic smoking is a known cause of lung disease. Other causes include genetics, infections, or compromised immune systems. Chemotherapy and radiation treatments for cancer can also contribute to lung disease. Sometimes the cause of lung disease is unknown.

Lung disease has many types, all of which affect your breathing. Here are some common lung diseases:

Chronic obstructive pulmonary disease (COPD) produces airway obstruction from damaged alveoli walls. Asthma inflammation narrows airways and blocks them. Idiopathic pulmonary fibrosis makes walls surrounding the alveoli become scarred and thickened. Lung cancer can start in your alveoli. Pneumonia makes alveoli fill with fluid, limiting oxygen intake. The normal aging process can slow down respiratory system, lung capacity is lessened, or chest muscles become weaker. Older people also tend to be more at risk for pneumonia, both bacterial and viral.”

WebMD.com states: “Bronchodilators are medications that relax muscle bands that tighten around airways. This opens the airway and lets more air move in and out of lungs. That helps breathe more easily. Bronchodilators also help remove mucus from your lungs. Open airways mean mucus can move more freely, too, and you can cough it up.

Short-acting bronchodilators are used as a “quick relief” or “rescue inhalers”, while long-acting bronchodilators can be used every day to control asthma along with an inhaled steroid.

For treating asthma symptoms, there are three types of bronchodilators: beta-agonists, anticholinergics, and theophylline. One can get these bronchodilators as tablets, liquids, and shots, but the preferred way to take beta-agonists and anticholinergics is inhaling them.

Short-acting bronchodilators are called quick-acting, reliever, or rescue medications, called rescue inhalers. These bronchodilators relieve acute asthma symptoms or attacks very quickly by opening airways. The rescue inhalers are best for treating sudden asthma symptoms. The action of inhaled bronchodilators starts within minutes after you inhale them and lasts for 2 to 4 hours. Short-acting bronchodilators are also used before exercise to prevent exercise-induced asthma“.

Mucus is important for our bodies, is like the oil in the engine. Without mucus, the human engine seizes. Elemental Life Solutions states: “Mucus-producing tissue lines the mouth, nose, sinuses, throat, lungs, and gastrointestinal tract. Mucus acts as a protective blanket over these surfaces, preventing the tissue underneath from drying out. Mucus also acts as a sort of flypaper, trapping unwanted substances like bacteria and dust before they can get into the body particularly the sensitive airways. It also contains antibodies that help the body recognize invaders like bacteria and viruses.” It also contains enzymes that kill the invaders it traps, protein to make the mucus gooey and stringy and very inhospitable, and a variety of cells, among other things. Yellow or green mucus is a clear sign of an infection, immune system sends white blood cells called neutrophils that contain a greenish-colored enzyme.

An article in the New England Journal of Medicine by John Fahy and Burton Dickey state: “The lungs are remarkably resistant to environmental injury, despite continuous exposure to pathogens, particles, and toxic chemicals in inhaled air. Their resistance depends on a highly effective defense provided by airway mucus, an extracellular gel in which water and mucins (heavily glycosylated proteins) are the most important components. Airway mucus traps inhaled toxins and transports them out of the lungs by means of ciliary beating and cough. Paradoxically, although a deficient mucous barrier leaves the lungs vulnerable to injury, excessive mucus or impaired clearance contributes to the pathogenesis of all the common airway diseases. The normal formation and clearance of airway mucus, the formation of pathologic mucus, the failure of mucus clearance that results in symptoms and abnormal lung function, and the therapy of mucus dysfunction.”

Pulmonary alveolar proteinosis (PAP) is a rare clinical syndrome that was first described in 1958. To date, whole-lung lavage (WLL) is still the gold-standard therapy for PAP. Herein, F. Gao, G. C. Lu, X. Y. Zhou, Z. Yu, H. M. Wang and T. Bien from Department of Respiratory Medicine, Wuxi People's Hospital Affiliated to Nanjing Medical University, Jiangsu, China in Genet. Mol. Res. 13 (3): 6135-6141 (2014), report the case of a male patient who was diagnosed with PAP by open-lung biopsy 8 years prior to presentation at our clinic. The man underwent his first WLL in 2004 and showed marked clinical and radiological improvement after the operation. However, after his original presentation, proteinaceous material continued to accumulate in his lungs. Lavage was performed four additional times, but these attempts failed to arrest the decline in pulmonary function. Each lavage resulted in significant, although transient, clinical improvement.

Pulmonary alveolar proteinosis is a disease caused by increased accumulation and impaired clearance of surfactant by alveolar macrophages. This narrative review in J Bronchol Intervent Pulmonol, Volume 22, Number 3, July 2015, www.bronchology.com, of Wolters Kluwer Health, Inc., summarizes the role of therapeutic whole-lung lavage in the management of pulmonary alveolar proteinosis. We describe the pre-procedural evaluation, indications, and anesthetic considerations, along with step-by step technical aspects of the procedure, postoperative recovery, potential complications, and long-term outcomes. PAP patients after WLL procedures typically use portable home pulse oximetry to wean themselves off supplemental oxygen if they ended up using it post-operatively, and follow-up with their pulmonologists in 2 weeks for an assessment. Smoking cessation and lifestyle modification may be important to maintain remission. Recently a single-center cohort study has shown smoking to be associated with an increasing number of WLL sessions to achieve remission.

In the U.S. Pat. No. 10,596,312, from Mar. 24, 2020, entitled “System for improving fluid drainage”, Hiemenz, et al teach a low-cost and simple-to-use system and method to facilitate a prophylactic pleural lavage protocol at the time of thoracostomy tube placement for traumatic hemothorax in order to reduce the need for secondary intervention for the management of retained hemothorax. The invention may be used in conjunction with existing chest tubes and be administered at the time of initial chest tube placement, and continued at the bedside (by a bedside nurse) over the duration of chest drainage, as necessary. The system includes an operator device that semi-automatically administers a pleural lavage protocol consisting of saline instillation, and suction to slow the clotting process, prevent “gelling” of blood, and maintain drainability. Compared to this patent our system improves the washing factor and allows more flexibility in treating the lung, under a quality assurance monitoring system.

In the U.S. Pat. No. 10,335,558, from Jul. 2, 2019, Boucher, et al. teaches some “Methods of treatment” directed to methods, compositions and apparatus for administering active agents to the lungs of a subject, which is a method of treating at least one lung/the lungs of a subject in need thereof, comprising: administering an active agent to the at least one lung/the lungs of a subject (for example, by sustained administering or infusion administering), using aerosol or inhalation administration.

An administering step is carried out by a nasal cannula, face mask, or positive airway pressure mask (e.g., a continuous positive airway pressure (CPAP) mask or a bilevel positive airway pressure (biPAP) mask), or by administration of the active agent to airway surfaces, in order to enhance mucus clearance from at least one lung of the subject.

An example of the invention is a method of enhancing mucus clearance from the lungs of a subject in need thereof, comprising: administering an osmolyte to airway surfaces of the lungs in an amount sufficient to hydrate said lung airway mucus secretions, and insufficient to substantially dehydrate lung airway epithelia cells therebeneath, said administering step being carried out and for a time sufficient to enhance mucus clearance from the lungs of said subject, or by administering said subject an aerosol comprising said osmolyte such as saline or hypertonic saline. An active agent as described herein in a pharmaceutically acceptable carrier (e.g., a liquid carrier, a dry powder carrier) for use in carrying out by an aerosol generator or nebulizer

The present inventions improve the application of medication only after alveoli were cleared from mucus, increasing the effectiveness of medication.

### SUMMARY OF THE INVENTION

The present invention is about a method to clean and repair lungs in order to increase the efficiency of the lung by an advancement in the current lavage process. The invention uses a multiple freedom degree bed to place the patient in various positions allowing gravity to aid the alveolar washing process with respect to operations of filing the lungs with the liquid agent, shaking and moving the lungs, and helping to drain the lungs and the treatment process.

This may be developed as an emergency lifesaving method for the illness induced by viruses as SARS, MERS, COVID-19, etc., that require alveolar cleanup, sanitizing, and healing. A large variety of solutions, temperatures of applications, and drugs can be applied directly on alveolar surface.

Liquid is different than air, and lung muscles are not efficient in performing the normal respiratory process with a lung that is partially filled with liquid. The auxiliary enclosure helps the patient perform breathing movements, by alternating positive and negative pressure on thoracic cavity, from the outside.

Inside the lung, complex multi-lumen tubes are introduced, with air bellows that seal the air ducts inside the lung after primary bronchus splits into secondary bronchi, allowing a segment of lung to be treated while the rest is fed with respiratory mixtures.

The system is a combination of equipment and computerized procedures that are developed to acquire the operational purpose fast and safe and under quality assurance procedures, Following the procedure treated patient should usually not require a ventilator anymore and using their own lungs, which are continuously monitored.

This system also collects bio-medical data, measuring the lungs lobe by lobe, imaging them by X-ray and ultrasound and immersed camera, obtaining 3D images, measuring the oxygen exchange efficiency, and analyzing the compositions on site and additionally in laboratory.

The lavage procedure has four washing stages:
clean the lung lobe from mucus and other depositions
kill the viruses and bacteria
heal alveolar wall and bronchi
apply enhancers and measure the functionality,

Due to the complexity of the system and the high sensitivity of the patient, where the dimensions are in the micron range with interactions of complex biochemistry and organism physiology, the parameters are very carefully adjusted to prevent any harm or damage. The patient's lungs are intended to be repaired and rendered functional rapidly after the affected zones are addressed.

This capability will stimulate research in developing new drugs and technologies in order to extend the success rate in treating incurable diseases and improving the performances even over what nature provided.

The system is a modular open system, and any supplementary required function may be added, or its complexity may be reduced to a minimum necessary.

### BRIEF DESCRIPTION OF THE DRAWINGS

FIG. 1 is a view of the actual method seen from a side;

FIG. 2 is a simplified view of the actual method seen from above;

FIG. 3 shows the schematics diagram of the fluid circuit

FIG. 4 details lavage technique

FIG. 5 describes a mobile operator bio-parameter monitoring system that is distributed on operator's body,

FIGS. 6A-F gives details on alveoli and bronchiolitis Pathophysiology:

FIG. 6A—Healthy alveoli

FIG. 6B—Alveoli and bronchi affected by bronchiolitis

FIG. 6C—Detail schematic view of an alveolar wall

FIG. 6D—Microscope image of alveoli;

FIG. 6E—Scanning Electron Microscope image (180×1) of lung;

FIG. 6F—Schematic diagram of forces inside lung;

FIG. 7 describes an operational room, with patient in near horizontal position;

FIG. 8 describes operational room, with patient in near vertical position;

FIG. 9 shows a section through the upper side of a body, in section through the center of the left lung;

FIG. 10—Cross section through the lungs;

FIG. 11—Schematic diagram of a fluidic modulus;

FIG. 12—Lavage tubes;

FIG. 13—Adjustable position in 3D patient bed, with lavage system mechanics and fluidics;

FIG. 14—System to hold the patient floating on the bed;

FIG. 15 Process control system with specialized data acquisition, and data integration with visualization;

FIG. 16—A system to measure and analyze patient bio-parameters, and integrate in process control systems;

### FIGURES DETAILS

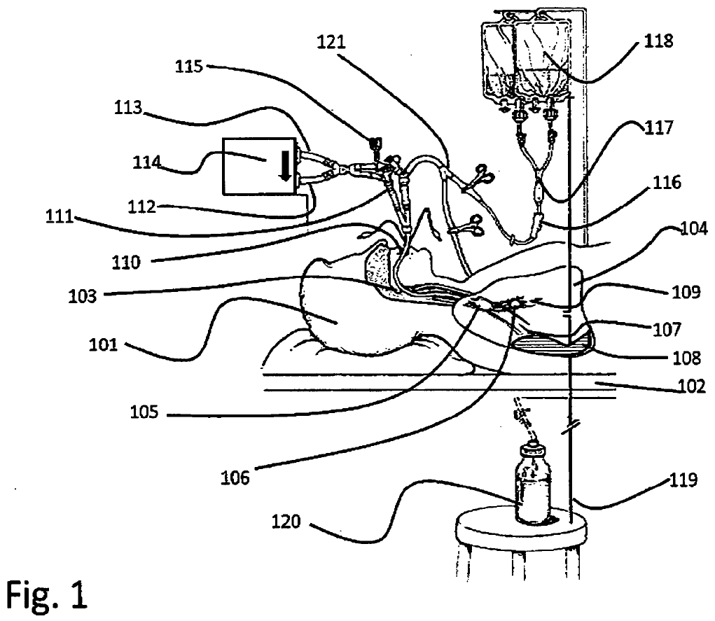

FIG. 1—is a view of the actual method seen from a side;
101—Patient placed on table;
102—Operational table;
103—Esophagus with lumens insorted by mouth;
104—Lung to be washed
105—Inflatable bellow in primary bronchus to seal the right lung;
106—Inflatable bellow in primary bronchus to seal the left lung;
107—Washing lumen;
108—Liquid deposition on the lower part of right lung;
109—Ventilated left lung;
110—Tube inserted through mouth;
111—Lumen mixer-splitter;
112—Pumping unit exhaust;
113—Pumping unit liquid input;
114—Pumping unit liquid flow adjustment;
115—Medical valves;
116—Infusion fluid flow adjustment;
117—Infusion fluid mixer;
118—Infusion fluid;
119—Medical fluid bags support.
120—Drain liquid collector, placed on a stool;
121—Flow splitter.

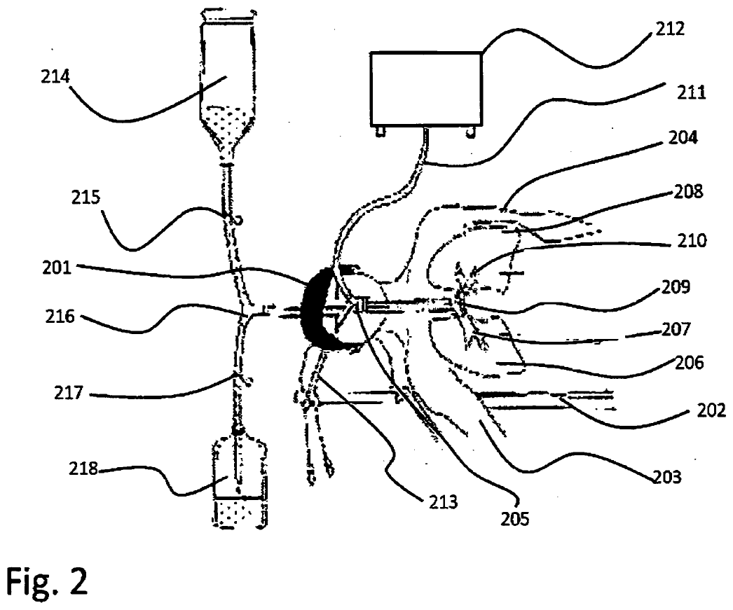

FIG. 2—A simplified view of the actual method seen from above.
201—Patient placed on table;
202—Operational table;
203—Right arm;
204—Left arm;
205—Tubes inserted in the mouth;
206—Right lung lavaged;
207—Primary right bronchus;
208—Left lung, ventilated;
209—Trachea;
210—Left primary bronchus;
211—Air tube;
212—Ventilator;
213—Aeration valves;
214—Lavage solution;
215—Medical on/off valve;
216—Mixer/router;
217—Drainage tube, medical on/off valve;
218—Drain bottle;

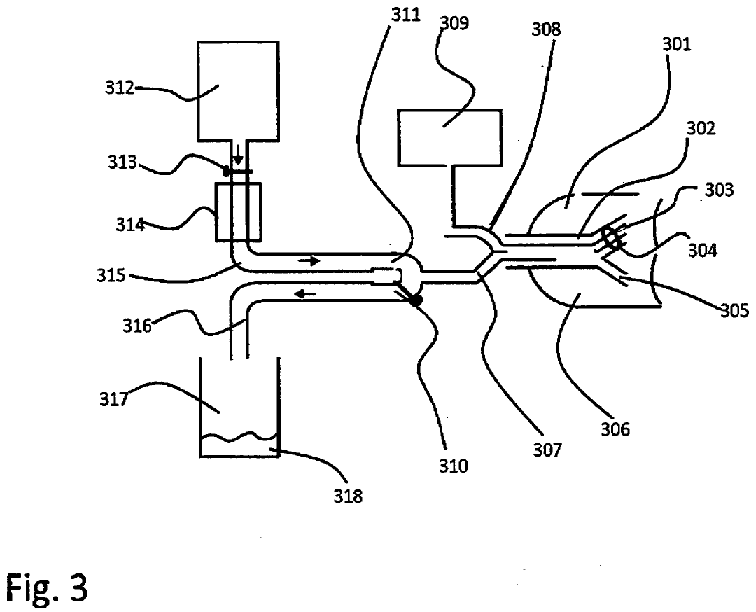

FIG. 3 shows the schematics diagram of the fluid circuit
301—Left lung, under ventilation;
302—Trachea;
303—Ventilation lumen;
304—Insolation cuff inflated;
305—Lavage lumen in the right bronchus;
306—Lavage left lung;
307—Liquid tube;
308—Air tube;
309—Ventilator
310—Drainage limb with lock;
311—Lavage limb;
312—Lavage fluid tank;
313—Fluid lock;
314—Fluid warmer;
315—Lavage fluid tube;
316—Drainage fluid tube;
317—Drainage fluid collector vessel;
318—Collected drainage fluid;

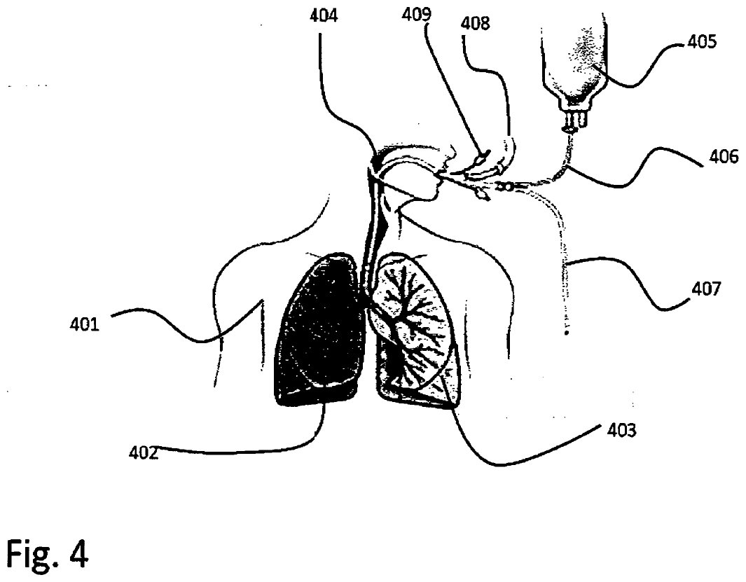

FIG. 4 details lavage technique
401—Patient
402—Right lavage lung
403—Left ventilated lung
404—Trachea with tubes inserted
405—Lavage solution bottle
406—Tube
407—Drainage tube
408—Air tube
409—Aeration, pressure limiter

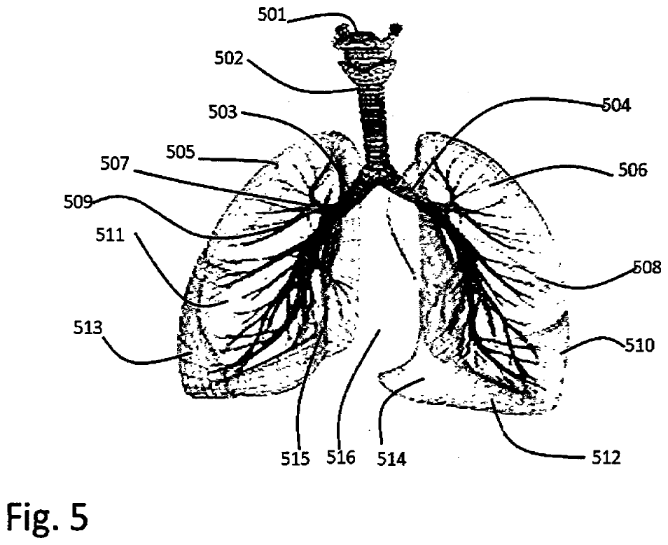

FIG. 5 describes the respiratory system
501—Larynx;
502—Trachea;
503—Right primary bronchus;
504—Left primary bronchus;
505—Right upper lung;
506—Left upper lung;
507—Right secondary bronchus;
508—Left tertiary bronchus;
509—Right tertiary bronchus;
510—Left bronchioles;
511—Right lung smaller bronchi;
512—Lower lung alveolar duct;
513—Right lateral lung alveoli;
514—Left internal lung alveoli;
515—Pulmonary artery right lung;
516—Cardiac notch;

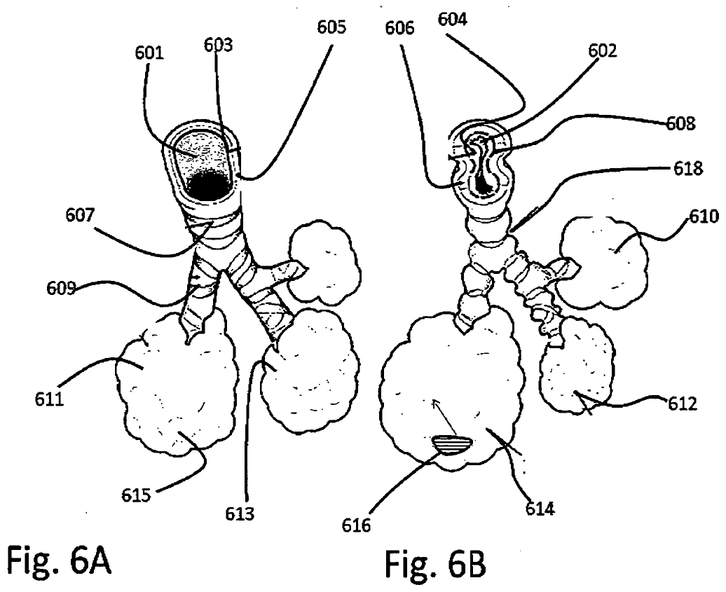

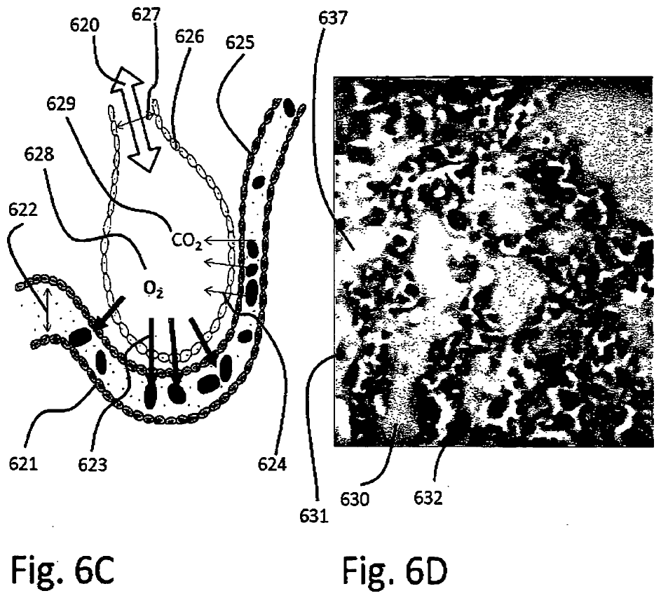

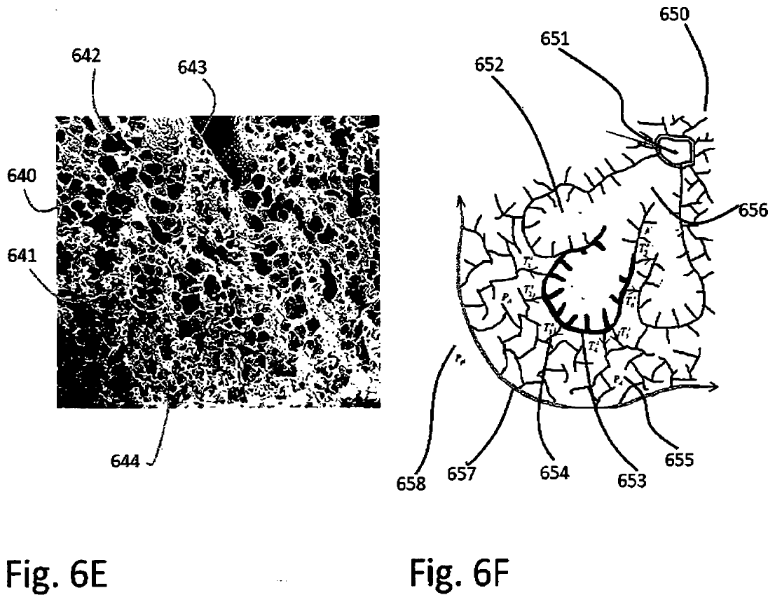

FIG. 6 gives details on bronchiolitis pathophysiology,

FIG. 6A—Healthy alveoli
601—Normal bronchial tubes;
603—Normal epithelium;
605—Normal tissue;
607—Smooth muscle, is tightening around
609—Bronchioles, making air penetration inside;
611—Alveoli, that are initially healthy;
613—Healthy alveoli;
615—Healthy alveoli lobe;

FIG. 6B—Alveoli and bronchi affected by bronchiolitis
602—Tubes during bronchiolitis, which are inflamed, clogged, with holes closed;
604—Border the micro-bronchial tube, becomes bended, covered by mucus buildup;
606—Inflamed tissue, that swallows,
608—Necrosis and lose of tissue, making air flow whistle;
610—Alveoli;
612—Alveoli affected and collapse having walls stick on each other due to mucus buildup;
614—Alveoli under treatment;
616—Liquid trapped with mucus;
618—Bronchial tubes, shrinking due to illness;

FIG. 6C—Detail schematic view of an alveolar wall
620—Bronchiole air micro-duct;
621—Bronchial wall;
622—Bronchial wall thickness, about 1 μm;
623—Oxygen diffusing in bronchial wall blood vessel trapped in a red cell;
624—Carbon dioxide diffusing through bronchial wall, outside the red cells;
625—Bronchial inner wall;
626—Alveolar wall;
627—Bronchiole opening diameter;
628—Oxygen molecule;
629—Carbon dioxide molecule.

FIG. 6D—Microscope image of alveoli;
630—Microscope image of alveoli;
631—Red blood cell;
632—Alveoli wall;
633—Bronchioli;

FIG. 6E—Scanning Electron Microscope image (180×1) of lung;
640—Bronchiole tubule;
641—Selected zone;
642—Alveoli cavity;
643—Alveolar wall;
644—Alveolar structure;

FIG. 6F—Schematic diagram of forces inside lung;
650—Schematic image;
651—Bronchiole;
652—Alveolar sac;
653—Alveolar wall;
654—Alveolar wall stress Tᵢⱼ;
655—Pressure inside alveoli;
656—Alveolar space;
657—Pleural membrane;
658—Extra pleural pressure;

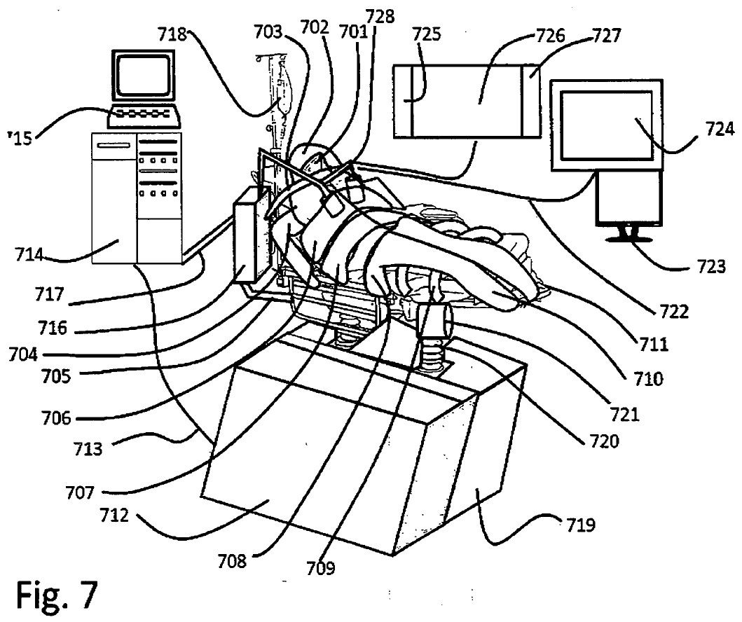

FIG. 7 describes operational room, with patient in near horizontal position
701—Patient's face under hyper-baric suite;
702—Hyperbaric helmet;
703—Patient respiratory and washing multi-duct
704—Patient's arm, on measurement devices;
705—Mobile bed—multi-freedom degrees:
706—Shoulder and torso hydro-pneumatic enclosure;
707—Abdominal hydro-pneumatic enclosure;
708—Basin and legs hydraulic support;
709—Leg immobilization and measurement bracket;
710—Inferior leg hydraulic support and massage device;
711—Bed infrastructure;
712—Power system for bed actuators;
713—Cable from computer control unit;
714—Computer unit for positioning control;
715—Computer keyboard and lung vectorization;
716—Utilities and fluids pumping box;
717—Cables from computer to imaging and auxiliary units;
718—Patient supplementary liquid system;
719—Auxiliary power and service systems;
720—Bed actuators;
721—Rotational gearbox actuator;
722—Cables to X ray 3D visualization unit;
723—Vibration, ultrasound and sound control and visualization unit;
724—Breathing, and lung measurement and simulation unit;
725—General procedure control menus;
726—General procedure control visualization unit;
727—Fluid control and lung performances assessment unit;
728—3D X ray unit for real time fluid control.

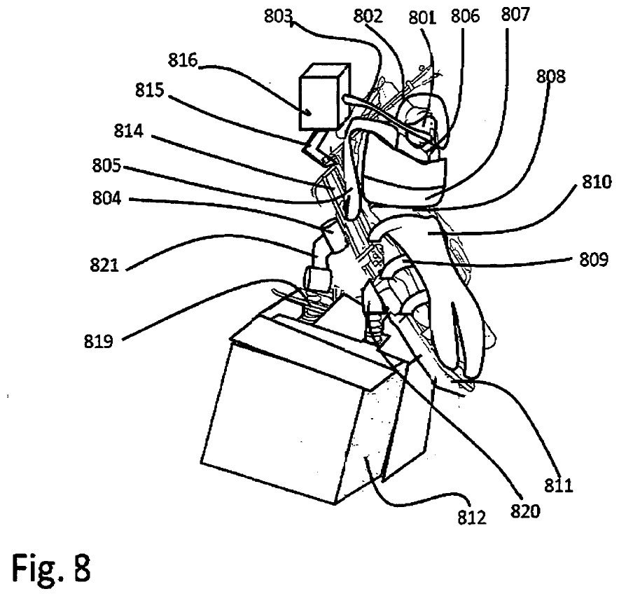

FIG. 8 describes operational room, with patient in near vertical position
801—Patient's face under hyper-baric suite;
802—Hyperbaric helmet;
803—Patient respiratory and washing multi-duct
804—Patient's arm, on measurement devices;
805—Mobile bed—multi-freedom degrees:
806—Shoulder and torso hydro-pneumatic enclosure;
807—Abdominal hydro-pneumatic enclosure;
808—Basin and legs hydraulic support;
809—Leg immobilization and measurement bracket;
810—Inferior leg hydraulic support and massage device;
811—Bed infrastructure;
812—Power system for bed actuators;
814—Bed position and pressure control;
815—Support arm and connections for utility fluid box;
816—Utilities and fluids pumping box;
819—Auxiliary power and service systems;
820—Bed actuators;
821—Rotational gearbox actuator;

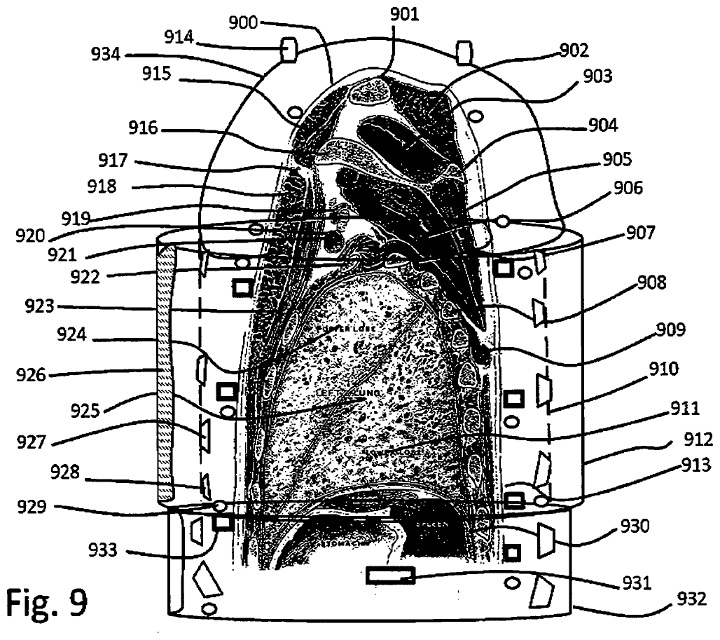

FIG. 9 shows a section through the upper side of a body, in section through the center of the left lung
900—Patient body;
901—Clavicle;
902—Trapezius muscle;
903—Supra-spates;
904—Spine of scapula;
905—Infra-supinates;
906—Subscapularis;
907—Serratus mangos;
908—Rib;
909—Rhomboids major;
910—Torso breading compressed air membrane and sound/ultrasound and vibration generator support;
911—Lung lower lobe
912—External torso hyperbaric tube;
913—Inner hydro-bag foil on skin contact
914—Upper arms and helmet hyperbaric seal;
915—Clavicular part of trapezius major;
916—Coracoid;
917—Cephalic vein;
918—Sternal part of pectoralis major;
919—Axillary artery;
920—Brachial nerves;
921—Axillary vein;
922—Rib ii.;
923—Pectoralis minor
924—Upper lobe;
925—Left lung lobes;
926—Compressed air bag for breathing and pressure regulation
927—Electromagnetic actuated membrane for vibration generation in audio and ultrasound spectrum
928—Phased array of vibration generators;
929—Longitudinal ultrasound visualization phased array;
930—Diaphragm;
931—Ultrasound generation phased array;
932—Belly region hyperbaric enclosure;
933—Ultrasound visualization phased array, placed along body;
934—Upper torso and shoulder support.

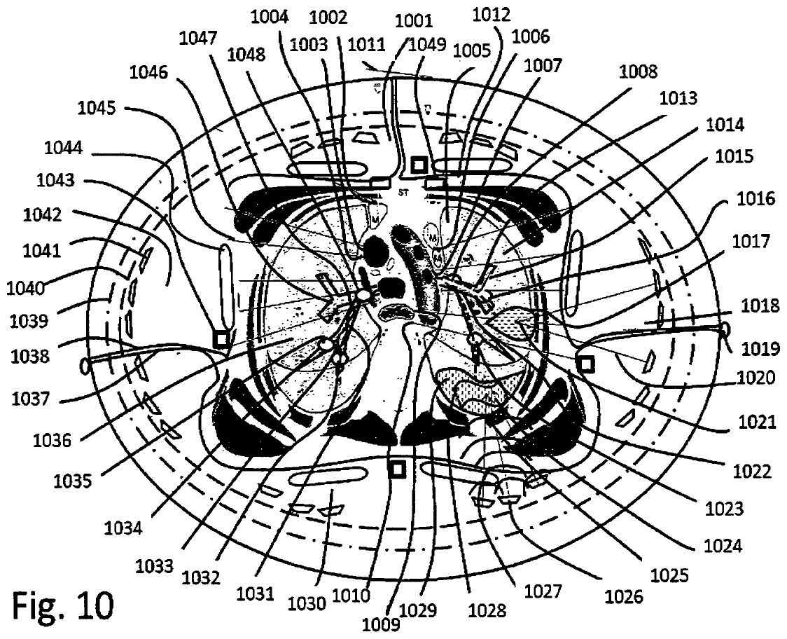

FIG. 10—Cross section through the lungs
1001—Patient body cross section at lungs median level;
1002—Internal Mammary Vessels;
1003—Superior Vena cava;
1003—Right phrenic nerve;
1004—Arch of Azygous;
1005—Right vagus nerve;
1006—Left phrenic nerve;
1007—Arch of aorta;
1008—Left lung primary bronchia near left vagus nerve;
1009—Esophagus;
1010—Thoracic duct
1011—External lock of thoracic hyperbaric enclosure;
1012—Patient's skin and sub-skin, fat tissue;
1013—Secondary bronchi;
1014—Upper left lung lobe;
1015—Tubule carrying gas to upper left lung alveoli;
1016—Secondary bronchi inflatable cuff plug;
1017—Liquid puddle accumulation in the alveoli;
1018—External liquid pressuring the body;
1019—Hinges at the outer containment;
1020—Pressure wave aiming in phased array;
1021—Focusing region to agitate the washing liquid;
1022—Tertiary bronchiole;
1023—Tertiary bronchiole cuff plug and multiple tubes pass through;
1024—Lavage liquid fulfilled alveoli;
1025—Directed pressure wave from a phased array;
1026—Electromagnetic to pressure wave transducer;
1027—Aiming direction of the pressure cardioid distribution;
1028—Lateral moving pressure wave;
1029—Upper alveoli partially filled with liquid;
1030—Ultrasound imaging phased array;
1031—Technological tube passing in secondary bronchiole;
1032—Micro tubes for light and analysis;
1033—Tubules passing in secondary bronchioles for oxygenation purposes;
1034—Tertiary bronchiole;
1035—Bronchiole sealing cuff, with technological tubule passage;
1036—Right upper lung on gas feed;
1037—Hydraulic separation membrane;
1038—Upper right side of hydraulic compression membrane;
1039—Inner cuff separation between the pressure and breathing volume and hydraulic medium;
1040—Support structure for electromagnetic to vibration transducers, hydrophones and ultrasound imaging arrays;
1041—Electromagnetic to vibration transducer;
1042—Intermediary liquid;
1043—Hydrophone;
1044—Ultrasonic imaging phased array;
1045—Patient inter-rib muscles;
1046—Tertiary bronchiole;
1047—Technologic tubes split;
1048—Secondary bronchiole insulation cuff;
1049—Hart defibrillator electrodes

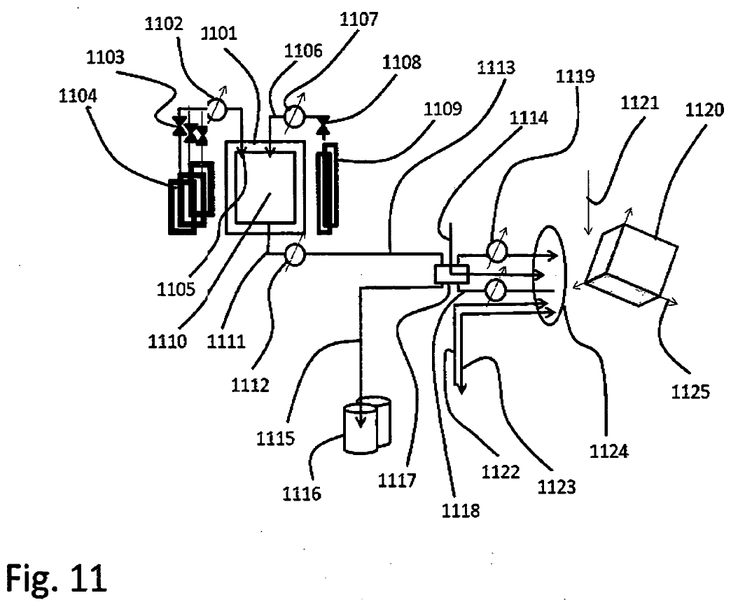

FIG. 11—Schematic diagram of a fluidic modulus
1101—Mixing unit;
1102—Pressure adjustment;
1103—Flow adjustment;
1104—Gas tubes;
1105—Gas mixture intake;
1106—Liquid mixture intake;
1107—Pressure adjustment;
1108—Liquid flow adjustment;
1109—Liquid tanks;
1110—Lavage fluid pump;
1111—Lavage fluid pipe;
1112—Pressure, flow, temperature adjustment;
1113—Lavage mixture delivery pipe;
1114—Measurement tube;
1115—Drain pipe;
1116—Drain tanks;
1117—Fluids valves block;
1118—Drain lavage fluid from lungs pipe;
1119—Input for lavage fluid with parameters adjustment;
1120—Patient and bed infrastructure;
1121—Gravitational field direction;
1122—Measurement input tube;
1123—Optical and drug delivery tubule;
1124—Secondary bronchi operation tube;
1125—Patient associated coordinate system for position control.

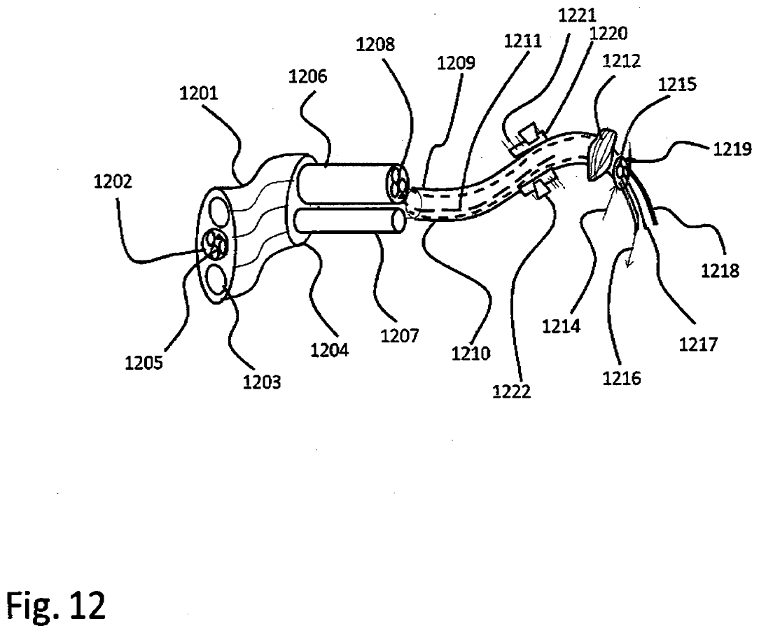

FIG. 12—Lavage tubes
1201—Tube of containment used from bed via helmet through mouth inside trachea;
1202—Tubule containing the tubes for one lobe's bronchiole lavage;
1203—Other lavage tube or breathing tube;
1204—end of central tube;
1205—Inner functional tubules;
1206—Tubule for bronchiole lavage;
1207—Breathing tubule;
1208—Respiratory mixture carrying tubule;
1209—Bronchiole tubule;
1210—External wall, containing steering channels;
1211—Left stirring string;
1212—Bronchiole sealing cuff;
1214—Drainage fluid flow;
1215—Drain tube;
1216—Respiratory gas filling flow on tubule;
1217—Optical cable;
1218—Measurement cable;
1219—Micro-imaging tubule;
1220—Myriapoda like advancement micro-motor;
1221—Translational spikes;
1222—Rotational spikes;

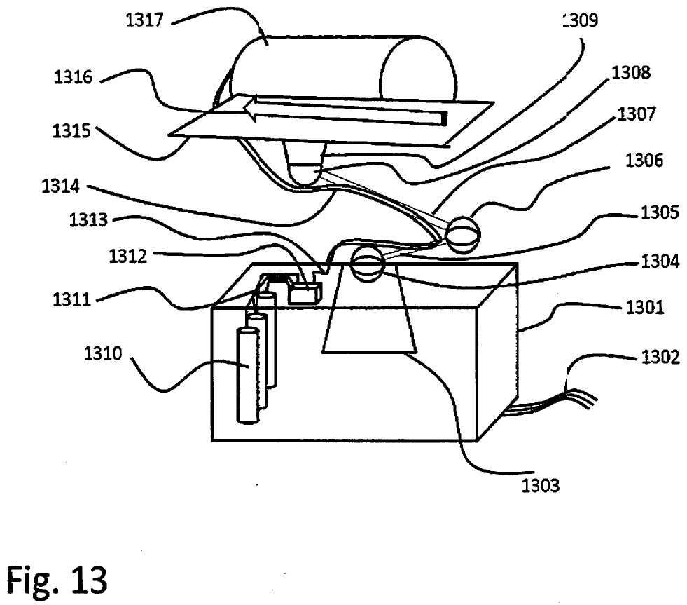

FIG. 13—Adjustable position in 3D patient bed, with lavage system mechanics and fluidics made of:
1301—Power actuators and position control box;
1302—Cables and fluidics power supply;
1303—Central arm turret;
1304—Bearing with gear and actuators for two freedom degrees;
1305—Telescopic inferior arm;
1306—Intermediary gear and actuator;
1307—Secondary telescopic arm;
1308—Final gear and actuator—bed turret;
1309—Two freedom degree articulation;
1310—Respiratory gas tubes
1311—Feed pipes to lavage solution preparation;
1312—Mixer and pumping system;
1313—Exhaust pipe to the central hose;
1314—Central hose, carrying fluids, measurement tubes;
1315—Bed structure;
1316—Bed positioning system;
1317—Patient lock in position cuffs;

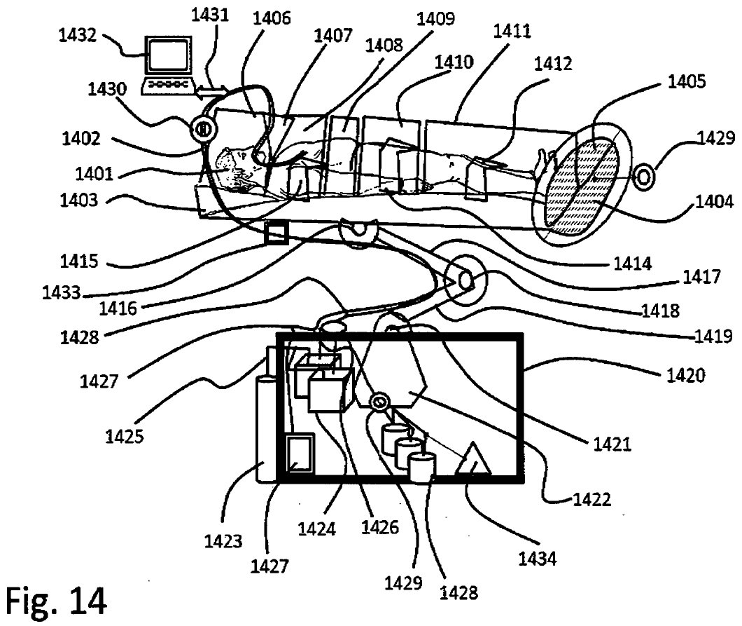

FIG. 14—System to hold the patient floating on the bed comprising:
1401—Patient, embedded into hinged cylinders;
1402—Hose containing technologic tubules;
1403—Patient's bed structure;
1404—A half cylinder with a half cuff fixed on the bed, operating as a water bed supporting the patient all along;
1405—Two quarter cylinders on lateral hinges that are surrounding the patient, making the flotation feeling, split over torso and abdomen also varying pressure for helping the patient breathing;
1406—Helmet structure;
1407—Mouth piece, passing technologic hose through helmet half cylinder covering the face, and accommodating the hoses that are inserted in the mouth and cable passage for instruments, being water and air tight;
1408—Thoracic tube and pressurized thoracic and abdominal cuffs;
1409—Abdominal compression tube;
1410—Hip compression quarter cylinders;
1411—Quarter cylinders covering basin legs, and arms for pressure equalizing;
1412—Leg safety brackets for setting patient secure in place;
1414—Hip safety brackets for setting patient secure in place;
1415—Arm safety brackets for setting patient secure in place;
1416—Bed turret and gear actuator;
1417—Upper arm with telescopic capability;
1418—Middle joint, actuator and gearbox;
1419—Lower telescopic arm;
1420—Power and hydraulics box;
1421—Lower turret gearbox actuator and lower arm joint;
1422—Actuator drivers and power box;
1423—Gas and liquids tubes;
1424—Lavage and respiratory fluid preparation and gas preparation units placed near the tanks;
1425—Connection fittings;
1426—Flowmeter, volumeter, thermometer, manometer measuring unit;
1427—A plurality of tubes carrying lavage liquids, joining in a common bunch to go up near articulated bed arm, to the helmet entry, and exhaust pipes going to liquid collector tanks;
1428—A plurality of lavage fluid drain tanks, for waste fluids recovery;
1429—A plurality of measurement instrumentation and control valves comprising a flow meter, volume meter, thermometer, manometer, sampler for laboratory analysis, optical spectrometry, and a sampling and measurement unit;
1430—Control system synchronized with patient, measuring pressures, temperatures, flow, volume, composition, etc., exhaust gas analyzer;
1431—Computer connection cable and data bus for process control;
1432—Process computer connected to data acquisition units;
1433—Vibration, Ultrasound and X Ray processing unit

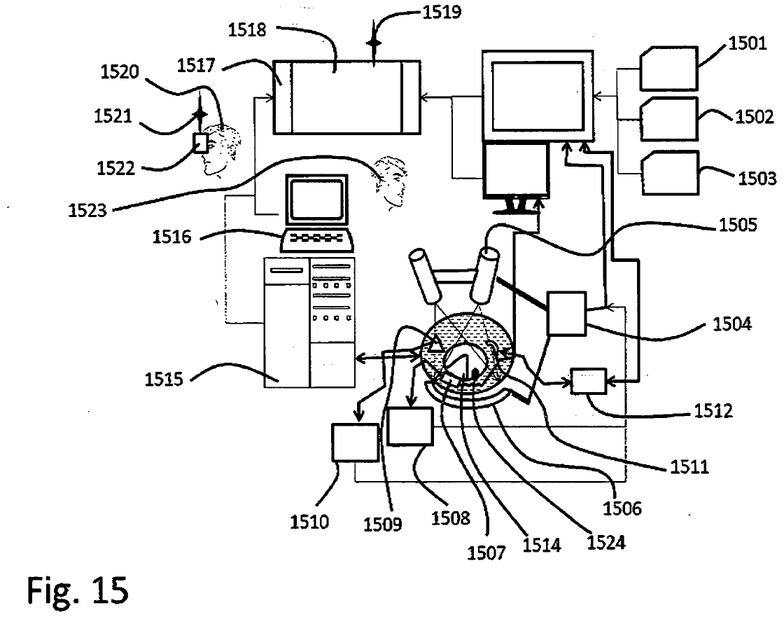

FIG. 15 Process control system with specialized data acquisition, and data integration with visualization
1501—Computer Tomography (CT) compatible data port and converters;
1502—Positron Emission Tomography (PET)) compatible data port and converters;
1503—Nuclear magnetic Resonance (MRI)) compatible data port and converters for display unit;
1504—A system to visualize inside lungs for stereoscopic X ray imaging;
1505—X—Ray generator tubes;
1506—Gantry arm, rotating around patient, for stereoscopic and CT modes;
1507—Ultrasound Phased Array (USPA) for imaging;
1508—USPA image processing box;
1509—Hydrophone receivers;
1510—Hydrophone data processing box for sound generator localization and sound partitioning;
1511—Electromagnetic Hydro-tweeter (EHT) for vibration waves generation;
1512—EHT control unit for phased array controlled washing pressure wave generation;
1514—Lung's lobe fulfilled with lavage liquid;
1515—Process control computer unit;
1516—Control panel and communication unit;
1517—Menu board on display unit;
1518—Lung immersion imaging unit;
1519—WiFi transmitter;
1520—Mobile operator;
1521—WiFi receiver;
1522—Augmented Reality (AR) display;
1523—Supervisor;

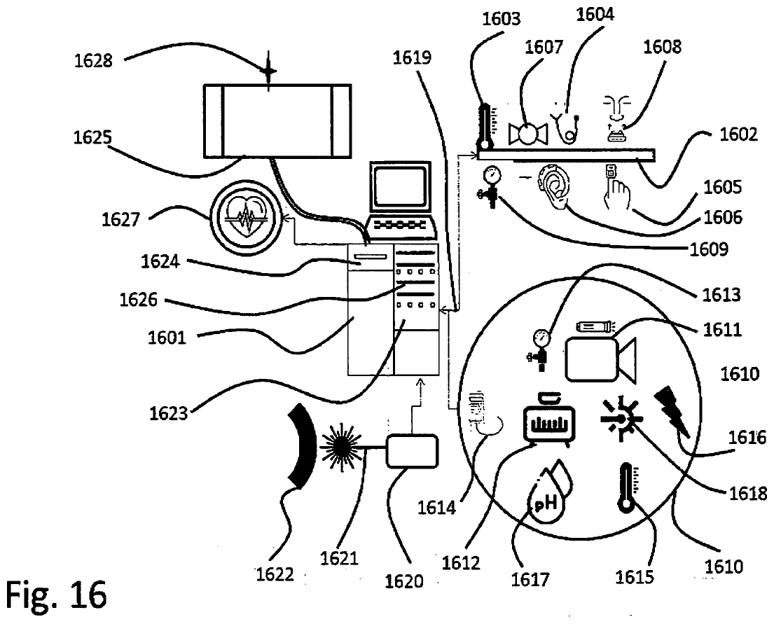

FIG. 16—A system to measure and analyze patient bio-parameters, and integrate in process control systems;
1601—A data acquisition system with computing simulation and visualization capabilities;
1602—A set or more, of wearable electronics placed in all compartments holding the patient measuring bio-medical parameters;
1603—Temperatures measurement in various locations;
1604—Pulse rate measurement;
1605—Oxygen concentration in blood measurement;
1606—Combined sensor for oxygen concentration, pulse rate and temperature measurement;
1607—Blood flow measurement by Doppler ultrasound, in exposed arteries as neck, arm, leg;
1608—Breathing air gas concentration measurement;
1609—Multiple pressure measurement sensors, for blood pressure on arms, and air pressure, other pressures in cuffs in real time;
1610—A system to measure inside lung parameters at the bronchiole level that may comprise many additional measurement and imaging devices;
1611—Video camera, with optic fiber illumination system;
1612—Optic fiber spectrometer
1613—Bronchiole pressure;
1614—Gas analyzers for each lavage tube ramification and breathing tube;
1615—Lavage temperature measurement;
1616—Lavage liquid conductivity measurement;
1617—Lavage liquids pH measurement;
1618—Laser for fluorescence spectroscopy measurement;
1619—Data bus connection to computer;
1620—Power IR laser;
1621—Fiber optic embedded in technologic lavage hose;
1622—Alveoli walls;
1623—Data fusion computer system for integrating all information;
1624—Communication interfaces with other process computers;
1625—Process visualization and control with quality assurance;
1626—Emergency procedures control with activation of emergency routines;
1627—Automated defibrillator;
1628—Emergency communication.

### DETAILED DESCRIPTION OF THE INVENTION

The inventors consider the developments in medical electronics, wearable electronics, hydraulics and pneumatics and medical lung therapy procedures. In parallel progress in aero-space technology, micro gravity flight simulators make possible revolutionary improvements in the lung lavage procedure, allowing high performance alveolar washing and treatment with fast recovery. One problem we intend to solve is to have a full controlled liquids management inside lungs, to provide liquid micro-agitation in order to wash better, without damaging the alveolar wall, but removing chronical depositions, virus and bacterial generated mucus, treat the cause and apply therapy procedures to make it better.

In order to provide a right therapy, it is good to have as much exploratory data in advance, but in emergency situations the system have to be able to extract those data as it goes, using bio-medical data acquisition system onboard.

It will come as a requirement or recommendation, to develop redundant communication systems as well as redundant power supply systems in order to be able to maintain a holistic knowledge and process control functions as long as possible during high severity events, or disasters, making the control process bring no contribution or to increase the impact factor, by aggravating the consequences/outcome of an accident or malfunction.

Having in mind that this system have to be operational in crisis time, too, that the ambient where augmented reality devices work, will have its own, redundant communication system, from, wire, optical to wireless, to local instruments, able to assure unperturbed operation. There are connected X ray stereoscopic imager, ultrasound phased array, that increases visualization depth inside the lung as much as it is fulfilled with fluid, due to propagation issues, in spongy environments, a sound analyzer/locator that may track the trachea sounds or defect alveoli falling sounds, laser spectral analyses, laser heating using micro-tubules and optic fibers, illuminating and imaging, UV/IR treatment, pressure, glow, temperature, gas content measurement and all necessary data processing and visualization.

We need the fluid suspension system, to assure a kind of micro-gravity like for the patient, obtained by his body buoyancy in water, but intend to have a dry environment, easy to be sanitized, and where the patient to be installed in minutes, and released in seconds in case of need, therefore water filled cuffs were used, that seal on patient body, being air tight.

It is there possible to use the patient suspension system as a hyper/hypo-bar enclosure, applying the treatments on a large range of pressures, from −0.5 bar up to 3 bar, if there is any reasonable purpose in doing this.

### BEST MODE OF THE INVENTION

FIG. 7 shows the devices in of the best mode contemplated by the inventors where the patient is positioned on the operational bed, connected to instrumentation, together with some solutions and developments that are embedded in the present invention.

The invention corrects previous deficiencies of the previous method, as follows;

a)—Improves the effectiveness of the lung lavage, by orienting the alveolar sac, together with the patient, similar to a bottle, in one position to be fulfilled with liquid and in other position to be emptied; shaking, rotating it such as to collect everything and take out, eliminating the need that to be absorbed via lung and removed as urine.

b)—Makes a system that allows operators a wide range of treatment procedures, from using a large variety of lavage mixtures, made from liquid and gases, and respiratory, healing mixtures, a large range of pressures, liquid gas agitation inside lungs, making possible not only diversification of existent treatments, but a research and continuous improvements in the field of medical lung research.

c)—Is easy, upgradeable being modular in structure, and having a virtual reality control room (one or several) upon the needs, and several augmented reality devices as wearables on operators, for real-time inside lungs immersive views;

d)—Has a complex sensor applicator on patient's body structure, easy removable, and with self-control of good operation, transmitting also patient's bio-parameters related to patient's state of wellbeing;

e)—Is redundant, have several internal pressure, temperature measurements, optical measurements, bio sampling from extracted liquids, mapping the lung and overlapping over 3D X ray image and ultrasound.

f)—Is developed in various functional approaches, from the threshold detection to anticipation, with different complexities and redundancy level, in agreement with the necessity, being more complex for a sick patient than for someone making a lung performance increase treatment;

g) Improves the medical personnel and patient access to a large variety of new treatment technologies, that may heal some patients in an exposure to this improved lavage technology, that may reduce medical equipment stress based on long term need of life assistance systems.

Best application of the invention is explained in FIGS. 7 and 8, but it is not limited to specific application presented and there are also some applications that do not require such complex equipment, and a simplified version is possible to be used, and gradually upgraded

### HOW TO MAKE THE INVENTION

It was necessary to build a similar device as air-control simulator seat, able to bring the pilot in any position relative to room coordinates, but in this case when replacing the cockpit with a hospital bed with a patient in, the only important acceleration that remained is the gravitational force, and in relation to this the entire positioning system is coordinated.

As FIGS. 5-6 shows the organ we address to treat is highly sensitive and vulnerable, and gravitational and inertial forces have to be carefully considered. This is aggravated by the fact that inserting liquids inside pulmonary lobes, makes liquid full alveoli weight more, stretching the lung internal structure, and we have to carefully move it such as to minimize the stretching due to weight. We need to fulfil with washing liquid, which we will like to behave as a foam, but not to fulfill the entire volume, and gradually wash the entire alveolar walls surface and go in the position to drain it outside the lung while leaving behind lung fulfilled by respiratory gas. The only purpose we added FIG. 6 is just to give a clear image of the difficulty of the operation and the degree of gentleness we have to act as to do the task with no harm inflicted.

FIG. 5 generally shows that we do not have many options, there are two lungs and 5 main lobes, and if we can lavage them one by one will be the limit of the actual technology, because it is difficult to introduce so many tubes through trachea and bronchi. Bronchiole level access will be chosen only in very special cases, where lobe level lavage is not recommended.

FIG. 6 A presents a healthy bronchiole and alveolar configuration, that will not be our primary work object, but the alveolar system showed in FIG. 6B which is what we have to treat and bring I the state shown in FIG. 6A.

In FIG. 6B, we show what is the level of lavage liquid, 616, we plan to introduce inside alveoli, just a little bit as to dissolve residues, wash the alveolar wall and train the residual liquid in th the drain tube, and for this we will rotate the entire body as to make liquid have a gravitational down flow, with as little turbulence as required for washing and training solid fractions.

FIGS. 5C and 5D shows how fine is the structure inside and how vulnerable, in a schematic view as well in an optical microscopic view, highlighting the accuracy we have to drive, because the alveolar wall has 1 micron in thickness, in order to allow osmotic diffusion of carbon dioxide in the fresh air, and oxygen from the fresh air having a concentration of about 20% in the blood to replace the carbon dioxide molecule which just diffused into air.

FIGS. 6E and F shows how intricate is inside the alveolar space, with emphasis on the fact that these 1 micron thick alveolar wall are carrying all the force and mechanical stress inside, which has to be limited to some moderate values to prevent ruptures and alveolar wall irreversible damage.

What we have to do, is just to treat that hyperfine structure without harming, by soaking first to soften or dissolve the solid and viscous agents, making them liquid effluents, drain that and then treat, rinse, and reinvigorate the structure, and finally measure and certify the quality of intervention.

How to accomplish the mission, is basically described I FIGS. 7 and 8. First we have to build a structure similar to a 3D flight simulator, able to place the pilot body in any position, that to be suitable for a hospital room, and have a bed instead of a cockpit with pilot's chair.

When one uses a flight simulator, bringing a pilot upside down is not a big deal, even if the general centrifugal movement stops and the pilot hang in the belts under his weight. Doing the same to a lung patient might trigger death, and the easy way to suspend a patient in any position is to use water generated micro-gravity by buoyancy, but immersing full body underwater requires tight sealing on respiratory system, and the presence of septic water over the entire body. Therefore, as FIG. 8 shows we still use water microgravity, but we pack the water into plastic bags set on the surrounding structure, that have a very tight contact with the skin al around the body, being dry, but making the function to sustain the body without stressing any part of it or organ. The problem of passing the respirator through the full body mask remains, but connecting and disconnecting is now faster, easier without contamination transfer from the infected body to surrounding liquid. Developing a such complexity system makes now possible the hypo-bar or hyper-bar treatments, that will create delays in patient respiratory tube mouth access because one have to weight that every pressure to normalize and opening the helmet to be safe. It is unclear at the very moment if from the medical point of view, there is any advantage on applying other pressures treatments and lung lavage, but because the lack of capabilities the medical studies have not been developed with an exception for healthy divers.

Due to this hydraulic developments there is now possible to rotate a patient inserted in a set of hydraulic tube sections, that add to the patient weight that may be up to 330 lb. (150 kg) adds another 150 kg of liquid, and the hardware it makes the weight that has to be rotated in space of about 500 kg (1100 lb.), which requires a strong mechanism in place, as shown in FIG. 12.

Another important technical development is the connection of the lavage tubes assembly to helmet and mouth-piece, where all the bunch of tubes have to penetrate through mouth into larynges than inside trachea and split on the 5 bronchioles as shown in FIGS. 10,11. It is desired that only one lung lobe to lavage at a time, the other four to be used for breathing, in normal or oxygen enriched atmosphere.

More, the desire is to use foamy solutions that not to completely fulfil the lobe's alveolar space, in order not to introduce too much weight and stress inside lung's structure showed in FIG. 6B.

Partially fulfilling the lung with lavage liquid, agitating the liquid inside using the pressure waves generated by electro-acoustic and vibration transducers immersed in the outside supporting fluid will, as seen in FIGS. 9, 10, contributes to lavage quality, and rotating patient's body in such a manner that all liquid to drain inside exhaust tub; will make the operation reliable and fast, with device described in FIG. 14.

The simplest way to perform the operation is to have the mouth-bronchiole piece as seen in FIGS. 11 and 12, with only two branches, one inside a lung lobe's bronchiole for lavage, and the other one sealing on trachea, for general breathing of the rest of the lobes. After each lavage procedure the helmet have to be opened in order to move the lavage section from a bronchiole to the next one, corresponding to the lung lobe to be lavaged, detailed in FIG. 9, as lateral view, and FIG. 10 as a cross section in lung as presented in FIGS. 3 and 4.

There is another way to do all at once, but in order to have lung spared of any interlobe contamination, but that requires a complex insert into bronchiole and trachea, acting as a rotary switch, more complex and different of what is the simple case presented in FIG. 12, with schematic fluidic circuit from FIG. 11, and it will be up to medical practitioners to evaluate the right technology, but for the very moment we think that a simpler system may be more reliable.

It is desired to perform measurements all along the process to assure that vitals and all operating parameters are good, and no risk for the patient may occur, and FIG. 15 and FIG. 16 are presenting briefly the bio-medical data acquisition which nowadays is modular and may be varied as function of need.

As patients' medical history varies, there are various lavage recipes that mainly provides a prewashing stage, with a liquid meant to dissolve, dislocate and fluidize the residues and solid deposits inside alveoli, than after the first content was drained out of lung, a second washing solution is applied, than a medical treatment solution and a final reinforcement solution meant to increase the lung's performances is applied. To assure the progress bio-parameters measurement is continuously performed. In order to increase the washing sounds and vibrations are used, directed by interference among multiple sources, used as a phased array towards the washed area as was presented in FIGS. 9 and 10. There are also ultrasound phased array introduced for imaging inside lung, as deep as possible, in order to detect the efficiency of the lavage procedures.

X ray and computed tomography, or stereoscopic 3D visualization may be obtained in real time but not all systems are needed.

The tubes may also have small radioactive sources in order to accurately localize their position inside lung, by using a goniometry process and also for density imaging, using internal point spectrometric source, similar to gamma radiography but performed with the radioactive source inside.

Modularity is desired for both method and equipment in order to be flexible and in any point to be able to add or remove a procedure or device to better serve the final goal, everything performed under quality assurance protocols, leaving no room for hazard.

### DETAILED DESCRIPTION OF THE FIGURES

The improvements of lung lavage processes, envisioned in this patent, starts from the idea that liquids already introduced inside lung alveoli are hard to be removed by the traditional manners based on blood absorption and removal by kidneys, where the micron size solid particulates remain and accumulates reducing the oxygen exchange capability and damaging the alveolar wall, triggering infections. The use of gravitational force turns out to be an easy solution to fill and drain a lung lobe, by gently rotating the body around gravitational vector direction, than refill and repeat the process until medical goals are obtained.

FIG. 1 is showing a view of the actual method seen from a side, representing the state of art at lung lavage technique. As a general medical practice, the patient, 101, under anesthesia is placed on table, 102 that may be a general purpose operational table. In order to insert the tubes for washing and ventilation, esophagus 103 is commonly used being more accessible for large tubes inserted by mouth. From esophagus 103, lumens inserted by mouth, 110, are accessing the right king to be washed, 104, and after reaching the right position in primary bronchus an inflatable bellow, 105, seals the right lung allowing the liquid to pour inside only, without leaking in the other lung.

In the left lung, the lumen carrying oxygenated air is sealed by an inflatable bellow, 106, inserted in primary bronchus of the left lung. In the right lung, washing lumen, 107, is now inserting the washing liquid, but due to gravitational field, a liquid deposition, 108, is occurring on the lower part of right lung.

All along the washing process, left lung, 109, is ventilated, using compressed air, oxygen, at normal or higher pressure. The operational tube, 110, is inserted through mouth containing several lumens, each performing a well-defined function.

The hydraulic circuit has the liquid source coming from several bags of infusion fluid, 118, most often a saline solution being used, placed on a medical fluid bags support, 119, and connected in parallel via an infusion fluid mixer, 117, into an infusion fluid flow adjustment, 116, being delivered to a flow splitter, 121. The flow direction is regulated using a pair of medical valves, 115, that may direct the flow through the drain tube towards the drain liquid collector, 120, placed on a stool, or towards the hydraulic pump, 114, passing through a lumen mixer-splitter, 111, towards the pumping unit liquid input, 113, that has adjustable flow, 114, and delivers it at pump's exhaust, 112,

FIG. 2 shows a schematic diagram of a simplified view of the actual method seen from above, where the patient, 201, is placed on an operational table, 202, with right arm, 203, and left arm, 204, placed along the body, with tubes, 205, inserted in the mouth.

Right lung, 206, is lavaged, by inserting the lumen in the primary right bronchus, 207, and inflating the cuff, while Left lung, 208, is ventilated, by inserting the lumen via trachea, 209, inside left primary bronchus, 210, using an air tube, 211, coming from a ventilator, 212, and having pressure limited by aeration valves, 213, that are also used to establish the clean right air concentration.

The lung's washing is made using, lavage solution, 214, usually made of 0.9% saline solution, that passes through a medical on/off valve, 215, to a “y” mixer/router, 216, that can switch towards lungs, or from lungs towards drain bottle, 218, via drainage tube, controlled by a medical on/off valve, 217.

FIG. 3 shows the schematics diagram of the fluid circuit used for lung lavage. The procedure keeps left lung, 301, under ventilation, by inserting ventilation lumen, 303, through trachea, 302, into left bronchus, inflating the insolation cuff 3041, in order to lavage left lung, 306, via a lumen, 305, inserted in the right bronchus.

A liquid tube, 307, is pouring a saline solution into the left lung, 306, while an air tube, 308, is introducing air from a ventilator, 309, into the right lung, 301.

The fluidic circuit is made of a lavage fluid tank, 312, that holds enough saline solution that flows gravitationally through a fluid lock, 313, into a fluid warmer, 314, that brings it to the right temperature exiting the lavage fluid tube, 315, into lavage limb, 311, that meets the drainage limb with lock, 310 that sets the flow direction towards the patient via the fluid tube, 307, when the lock, 310, is closed, or to the drainage fluid collector vessel, 317, via a drainage fluid tube, 316, accumulated as collected drainage fluid, 318, mixing with the fluid extracted from the patient.

FIG. 4 describes details lavage technique, where the patient, 401, has lavage on right lung, 402, while left lung, 403, is ventilated, by using his trachea, 404, to insert tubes.

The lavage solution is set in a bottle, 405, on a support, flowing down via a tube, 406, meeting the joint with drainage tube, 407.

The air tube, 408, is also introduced in trachea, having remained outside patient's mouth aeration, pressure limiter, 409, extensions.

FIG. 5 describes a respiratory system as a main importance for our patent, without being an embodiment of the present invention, because it represents the subject of the treatment method and apparatus that are developed to sustain the method. It is the object of our patent made of larynx, 501, connected to trachea, 502, that splits going to right primary bronchus, 503, and to left primary bronchus, 504, that further splits in right secondary bronchus, 507, and left, than splits again in left tertiary bronchus, 508, and right tertiary bronchus, 509, then splits more in left bronchioles, 510, and right lung smaller bronchi, 511, ending in Lower lung alveolar duct, 512, that drives into alveoli, no matter Ware right lateral lung alveoli, 513, or left internal lung alveoli, 514, they are about the same all over the lung and refurbishing them is the goal of our patent.

The lung is also divided in parts named after their position in the standing up body, as right upper lung, 505, and left upper lung, 506, as an example, but no matter where the alveolus are they are fed with blood coming from pulmonary artery 515, carrying out oxygen and veins bringing carbon dioxide to lung.

FIG. 6 are important because are describing in the finest detail the object of the treatment addressed by the patent, highlighting of the fines of the structure, that allows very little margin of error in the control of the lavage process.

FIG. 6A gives details on bronchiolitis pathophysiology by comparing normal bronchial tubes, 601, with a normal epithelium, 603, a normal tissue, 605, smooth muscle, 607, around bronchioles, 609, making air penetration inside alveoli, 611, and healthy alveoli, 613, 615. This shows a healthy lung lobe, and represents the lung desired shape after the treatment.

FIG. 6B shows in the finest details a sick tissue, with the tubes during bronchiolitis, 602, which are inflamed, clogged, with holes closed.

During illness a normal epithelium, 603, that border the micro-bronchial tube, becomes bended, covered by mucus buildup, 604, making the pressure drop grow, and leave less air inside.

The normal tissue, 605, becomes inflamed, 606, swallows, and sometimes is affected by necrosis and lose of tissue, 608, making air flow whistle.

Smooth muscle, 607, is tightening around bronchial tubes, 618, shrinking them, and the same around bronchioles, 609, making air penetration inside alveoli, 610, 611, very difficult. Healthy alveoli, 613, are affected and collapse, 612, having walls stick on each other due to mucus buildup.

Other alveoli, that are initially healthy, 611, may remain overinflated with trapped air, 614, due to collapsing of lateral lobes. Inside overinflated alveoli, 614, liquid may be trapped, 616, that is hard to be eliminated, and as normal lung mechanism it is transferred into blood, that carries it to kidneys and so it is eliminated, but hydrophilic mucus does not allow this process to take place, and lung remains out of use. In order that liquid trapped in a certain alveoli to be released, the lung have to turn around until the arrow pointing direction to bronchial orifice, 616, is align with gravitational field's direction. This is the typical tissue that will be the subject of patent application, where a sick lung tissue as shown in FIG. 6B have to be refurbished and brought at the shape in FIG. 6A, successfully dealing with all the manifestations of the disease.

FIG. 6C shows a detail schematic view of an alveolar wall, which is very fragile. A bronchiole air micro-duct, 620, bordered by bronchial outer wall, 621, with a thickness, about 1 μm, 622, between bronchial inner wall, 625, and outer wall, 621, where in between red cells are carrying carbon dioxide diffusing through bronchial wall, outside the red cells, 624, through alveolar wall, 626, into bronchiole with opening diameter, 627, releasing carbon dioxide molecule, 629.

Oxygen molecule, 628, in excess is diffusing in bronchial wall blood vessel and gets trapped in a red cell, 623, liberated from CO2 that diffused into alveolar space to balance the partial pressure distribution. One has to note the fine construction of the alveolar device, very sensitive to mechanical stress while it is as robust as possible made by nature.

FIG. 6D shows a microscope image of alveoli, 630, where red blood cell, about 400 nm size, 631, is drifting inside alveolar wall, 632, and pushing air into bronchiole, 633, that have small sizes in tens of microns, 637, overlapping in image due to very thin separation walls.

FIG. 6E—Scanning Electron Microscope image (180×1) magnification of lung, bronchiole tubule and alveoli, 640, where a selected zone, 641, have been selected for an in depth mechanical analysis. One may see an alveolar cavity, 642, bordered by a thin alveolar wall, 643, where alveolar structure, 644, is very dense, and complex.

FIG. 6F shows a schematic diagram of forces inside lung, with a zone extract, 650, from the nearby image, in a schematic representation, having in upper corner a bronchiole, 651, driving air into a bunch of alveolar sacs, 652, bordered by alveolar walls, 653.

Alveolar wall stress Tᵢⱼ, 654; is given by the pressure difference inside alveoli, 655, that determines the modification of the, 656, alveolar space, by the action of pleural membrane, 657, extra pleural pressure, 658, determined by inter-rib muscles action.

FIG. 7 gives a description operational room, with patient in near horizontal position, an embodiment of the present invention, where patient's face, 701, is seen under hyper-baric suite, that maintains same pressure from −0.5 bar to 3 bar on whole patient's body, in order to reach the best treatment condition under the aspect of pressure and temperature. The hyperbaric helmet, 702, has electronic visualization and monitoring equipment included, and allows the passage of patient respiratory and washing multi-duct, 703, that is an assembly of various dimension tubes that are introduced via larynx into patient's trachea and bronchi.

Patient's arm, 704, is included inside a pressurizing cuff, and contains various bio-medical parameter measurement devices, as blood pressure, pulse rate, oxygen content in fingers' blood vessels, etc., and is part of the hyperbaric system.

Patient's bed, 705, is mobile, mounted on a system with multi-freedom degrees, able to place the patient in any desired position, relative to gravitational field, in order to assure the fulfillment and drainage of the lavage liquid from any alveolar path, under the effect of gravity and vibrations from, electromagnetic to pressure wave transducers placed outside the patient's body, but inside hyperbaric enclosure.

Patient's shoulder and torso are placed inside the segment of hydro-pneumatic enclosure, 706, that is continued with an abdominal segment, 707, of the hydro-pneumatic enclosure, followed by hips and legs hydraulic support, 708, that provides legs immobilization and measurement via a bracket, 709 and also has included an inferior leg hydraulic support and massage device, 710, all rigid connected to bed's infrastructure, 711. The reasoning behind this complex structure was to provide a dry environment for the patient, able to use fluids for pressure wave applications and ultrasound imaging, as well to provide a massage for circulatory purposes, in limbs, everything easy and fast to open, and release the patient. It is also important that no part of the patient's body to be left outside of the controlled pressure area, because that may drastically affect patient's safety.

A hydro-proof power system, 712, for bed actuators, which is connected by cables, 713, to a computer control unit, 714, for bed's positioning control. The operator, using computer's keyboard, 715, may perform lung's alveoli location and vectorization, such as the fluids, gases and liquid mixtures, coming from utilities and fluids pumping box, 716, to act in harmony with gravitational forces at alveolar level, to fulfil it with liquid, when the alveoli is placed under the filling tube, and get empty when the body is rotated by the bed actuators until the alveoli is placed above the bronchi and extraction tube, all content leaking towards the outtake tube. Body rotation is used such as the lavage liquid to gently wash all alveolar surface, being agitated by the pressure wave, that improves the cleaning of the alveolar walls. The lavage liquid is controlled in pressure, temperature, chemical content, gas mixture and flow, such as to maximize the cleanup effect. It will be possible to have some inner visualization by micro camera, laser illumination and fluorescence spectral analysis, as well to transport power radiation on absorption bands of the targeted lung infestation agents, to supplementary warm them up. This equipment now base on the usage of few saline and antibiotics doped lavage solutions will open the way for intensive new research.

The local computer also receives imaging and analytical signals, that are further sent via cables, 717, from computer to imaging and auxiliary units that are specialized in generating 3D ultrasound images, or stereoscopic X ray images, generating immersive virtual reality for operator, helping operator to control tubes placement as well the lavage process. Other equipment is measuring the efficiency of alveolar membrane gas exchange monitoring the oxygen and carbon oxides concentrations, as well other gases specific to the lung expectoration materials being cleaned.

At the beginning, normal pressure operation, a patient supplementary liquid system, 718, may be used, but as soon as hyperbaric treatment starts, all pressures have to be controlled by an auxiliary power and service system, 719, that also powers bed actuators, 720, rotational gearbox actuator, 721, correlating pressures with patient's position, in order to avoid any internal undesired stress.

The quality of lavage procedure is assured using 3D stereoscopic X ray visualization system, where the information is transmitted by cables, 722, from X ray devices to 3D visualization unit, that integrates it with vibration, ultrasound and sound control and visualization unit, 723, and breathing, and lung measurement and simulation unit; 724, and sends them as virtual and augmented reality to operator's helmet.

Operators, may also access on augmented reality helmets, or on screens, general procedure control menus, 725, general procedure control and visualization unit, 726, fluid control and lung performances assessment unit, 727, and 3D X ray unit for real time fluid control, 728, integrated in a holistic process control and quality assurance.

FIG. 8 shows a describes operational room, with patient in near vertical position, that is appropriate for flooding lower lung lobes with lavage fluids, or drying upper lung lobes and taking out the washing liquid. The patient's face, 801, is under hyper-baric suite, wearing a hyperbaric helmet, 802, that allows patient's respiratory and washing multi-duct, 803, tube to be applied, and connected.

Patient's arm, 804, is inside a hyperbaric enclosure and is on measurement devices, for blood pressure, heart rate, oxygen concentration.

The patient is installed on a mobile bed, 805, having several/multiple freedom degrees, that allow it to place patient orientation in any position against gravitational field, and continuously pass through various position such as to assure the complete leakage of the washing fluid from any alveoli, no matter how intricate the bronchiole is. The patient shoulder, 806, and torso are placed inside a hydro-pneumatic enclosure, another one contains the abdomen, 807 than is followed by basin and legs hydraulic support, 808, that also has leg immobilization and measurement brackets, 809, and contains an inferior leg hydraulic support and massage device, 810, all fixed tight on bed's infrastructure, 811.

The bed is moved by a power system, 812, for bed actuators, 820, controlled by a bed's position and pressure control system, 814, and uses a rotational gearbox actuator, 821. A support arm and connections, 815, for utility fluid box, 816, which contains inside fluids pumping box, powered by an auxiliary power and service system, 819, that contributes to the success of the procedures.

FIG. 9 shows a section through the upper side of a body, 900, in section through the center of the lung. In the upper side one may see the clavicle, 901, covered by trapezius muscle, 902, and other supra-spates, 903, placed near spine of scapula, 904, and infra-supinates, 905, that are placed above subscapularis, 906, and serratus mangos, 907, making that direction inappropriate for imaging or vibration transmission purposes, but being a strong area that may be used for liquid pressure adjustments, in the patient weight support devices.

Ribs, 908, are surrounding the thoracic cavity therefore we have to accommodate their presence and rhomboids major, 909, ink, imaging systems and vibration application systems.

On the upper front there are clavicular part of trapezius major, 915, coracoid, 916, cephalic vein, 917, sternal part of pectoralis major, 918, axillary artery, 919, brachial nerves, 920, axillary vein, 921, ribs, 922, and pectoralis minor, 923, above and covering the upper lung lobe, 924, making the access from this direction difficult, too.

The technologic part of breathing assistance comprises a torso breading compressed air membrane and sound/ultrasound and vibration generator support, 910, contained inside an external torso hyperbaric tube, 912, that adjusts the pressure applied on whole body in harmony with the other units, and in order to apply fluid pressure while the patient remain dry it has a inner hydro-bag foil on skin contact, 913, that is permeable for ultrasound, vibrations and X rays leaving a good visualization and vibration energy transmission to lung lower lobe, 911.

Upper arms and helmet hyperbaric seal, 914, is connecting air tight to the upper torso and shoulder support, 934, that helps maintain the body head down in liquid flotation like suspension, by adjusting pressure inside.

Left lung lobes, 925, section is showed in the drawing, and the breathing is made by diaphragm movement, 930, and torso assisted compression synchronous with the diaphragm, that is helped by belly region hyperbaric enclosure, 932, which is making the pressure uniform along the body, and is contributing to the breathing process. Because human body is a fluidic system, hyperbaric pressure has to be applied uniformly over whole body in order to prevent tissue damage and blood dislocation.

A compressed air bag, 926, for breathing and pressure regulation is helping with fast response, and is lighter than water, moving up and down less than ½ psi. Inside the water bag are immersed electromagnetic actuated membranes, 927, for vibration generation in audio and ultrasound spectrum, working as phased array of vibration generators, 928.

For imaging purposes in nearby vicinity of the ribs, longitudinal ultrasound visualization phased array, 929, is used being aware of weak propagation through lungs, inflated with air, and gooey covered membranes, where ultrasound generation phased array, 931, and ultrasound visualization phased array, 933, are placed along body. Sound and sound time of flight is also received in electromagnetic transducers, 929, in part placed near the skin separation membrane, analyzing sound coming through liquid, if that lung lobe is flooded with lavage liquid, or through air, indicating the areas with problems, the physicians have to focus next.

FIG. 10 show a cross section through the lungs of patient body, where the cross section is performed at median lungs level, 1001, patient's skin and sub-skin, fat tissue, 1012, as patient is on its back, with internal mammary vessels, 1002, upwards and underneath is superior vena cava, 1003, and immediately is right phrenic nerve, 1003, and arch of Azygous, 1004, near right vagus nerve, 1005. On the other side one may see left phrenic nerve, 1006, arch of aorta, 1007, left lung primary bronchia, 1008, near left vagus nerve, enumerated just to show, the complexity, asymmetry and high technological level one have to possess to operate seamless in that vicinity.

We intend to use esophagus, 1009, thoracic duct, 1010, external lock of thoracic hyperbaric enclosure, 1011 to insert the tubes, via secondary bronchi, 1013, upper left lung lobe, 1014, for a tubule carrying gas to upper left lung alveoli, 1015, sealing them air tight through secondary bronchi inflatable cuff plug, 1016, and producing liquid puddle accumulation in the alveoli, 1017, that affects breathing In order to support the body, we use a big cuff with external liquid pressuring the body, 1018, that is made in several segments connected by hinges at the outer containment, 1019, such as to be easy to introduce the patient inside, and take him out dry.

Pressure wave aiming in phased array, 1020, is used to be focusing in lavage region, 1021, to agitate the washing liquid inside the alveoli, to improve the washing factor.

The idea is to produce lavage only in small sections of the lung, by using tertiary bronchiole, 1022, sealed by a tertiary bronchiole cuff plug, 1023, through multiple tubes are passing through, some bringing liquid mixtures for lavage liquid fulfilled alveoli, 1024, that is agitated by directed pressure wave from a phased array, 1025, that uses a plurality of electromagnetic to pressure wave transducer, 1026, a kind of underwater tweeter-loudspeaker, aiming direction of the pressure cardioid distribution, 1027, making a lateral moving pressure wave, 1028, by gently tuning the phase shill and transducers' orientation, in order to wash upper alveoli partially filled with liquid, 1029. As everything from pleura to alveoli is filled with liquid, ultrasound may propagate without significant attenuation, and an ultrasound imaging phased array, 1030, may be used to see in depth, much better than in a radiographic image. For more complex inside analysis, technological tube passing in secondary bronchiole, 1031, may have a camera, for inside imaging, while a micro tubes is used for light and spectral analysis, 1032, in various modes, as Raman, fluorescence, absorption and reflection bands, and other tubules passing in secondary bronchioles for oxygenation purposes, 1033, may also carry other gases, used for therapeutic or antibacterial purposes.

A tertiary bronchiole, 1034, is used, after being plugged with a bronchiole sealing cuff, 1035, with technological tubule passage, for multiple purposes among which measuring the oxygen exchange efficiency of the lobe, before and after the procedure application.

While a part of the left lung lobe is on lavage, right upper lung, 1036, is on gas feed, breathing normally or in oxygen enriched atmosphere, being assisted by the hydraulic system.

Upper right side of hydraulic compression membrane, 1038, has several hydraulic separation membranes, 1037, and an inner cuff, 1039, for separation between the pressure and breathing volume and hydraulic medium, that also houses inside a support structure, 1040, for electromagnetic to vibration transducers, hydrophones and ultrasound imaging arrays, electromagnetic to vibration transducer, 1041, hydrophone, 1043, array, localizing sound generated by air movement in lungs, ultrasonic imaging phased array, 1044, working together to assist the lavage process diagnose interior of the lung, alveolar walls, and more.

Because, patient inter-rib muscles, 1045, are most often weakened by disease, an intermediary liquid; 1042, is used, to assist breathing, by gentle pressure variation in synchronism with patient's diaphragm, to make possible breathing even if the lung is fulfilled with liquid by tertiary bronchiole, 1046, using complex technologic tubes split, 1047, from the secondary bronchiole insulation cuff; 1048, that is air tight, in order to control liquid separation from gas, and insulate the lavage lobe, until the entire restoration procedure is accomplished. For extreme situation, of cardiac arrest, a hart defibrillator is connected to specialized electrodes, 1049.

All the system is developed and operated giving in mind the complexity and fine operation in place aiming to restore functionality in lobe walls 1 micron thick.

FIG. 11 shows some fluidic circuit details, in a schematic diagram of a fluidic module, that includes a mixing unit, 1101, with pressure adjustment, 1102, and flow adjustment, 1103, of the fluid coming from gas tubes, 1104, preparing a gas mixture sent to intake, 1105, where meets liquid mixture intake, 1106, to mix them with liquid coming from a pressure adjustment, 1107, and liquid flow adjustment, 1108, from various liquid tanks, 1109, to prepare a complex gas-liquid lavage fluid.

A lavage fluid pump, 1110, takes the mixture, and adjusts the fluid pressure, flow function, delivering it into lavage fluid pipe, 1111, were a supplementary pressure, flow, temperature adjustment, 1112, is made and fluid goes into lavage mixture delivery pipe, 1113.

An advanced measurement tub; 1114, is measuring the liquids parameter inside lung.

After lavage, the patient body, is rotated from a position where trachea was superior to lung's lobe in inverse position, such as lavage liquid to drain into trachea that becomes inferior to alveoli sachet, and from there in drain pipe, 1115, to drain tanks, 1116. A fluids valves block, 1117, is making all the valves switches to guide the drain lavage fluid from lungs pipe, 1118.

Input for lavage fluid with parameters adjustment, 1119, is the final control for the lavage fluid before entering the patient's lungs, that is placed on a bed infrastructure, 1120, that has a patient associated coordinate system for position control, 1125, able to align a secondary bronchi operation tube, 1124, containing a measurement input tube, 1122, and an optical and drug delivery tubule, 1123, to gravitational field direction; 1121, such as the liquid naturally flow in and out alveolar sac.

FIG. 12 shows lavage tubes where a tube of containment, 1201, is used from bed via helmet through mouth inside trachea, and each tubule, 1202, is containing the tubes for one lobe's bronchiole lavage, and other few lavage tubes or a breathing tube 1203, going up to the end of central tube 1204.

A trachea tube contains a set of inner functional tubules, 1205, are carrying all necessary equipment for dedicated operations as tubule for bronchiole lavage, 1206, breathing tubule, 1207, respiratory mixture carrying tubule, 1208, bronchiole tubule, 1209, that are splitting, being introduced each in a bronchiole of the lung's lobe is desired to be washed or in meant to maintain body's oxygenation during the process. After lavage was performed at the desired lung lobes the tubes are withdrawn and a new trachea tube is introduced to lavage the rest of the lung, if the lavage process is designed to be done in one session.

Each tubule has an external wall, 1210, that incorporates stiff steering channels, and inside channels there are stirring strings, one or more left stirring string, 1211, and others for all directions, that act up to bronchiole sealing cuff, 1212 that when inflating has the role to seal the lung-lobe, and allow the lavage procedure to be performed without influencing other parts of the lung. The tubule ends after the cuff separating into more functional tubules, as one for drainage fluid flow, 1214, that have been used for washing, and is directed in the drain tube, 1215.

Nearby there is as a longer tubule a respiratory gas filling flow on tubule, 1216, that brings a mixture of gas and lavage liquid, that is aimed to certain group of alveoli. Care have to be taken to the amount of liquid loaded in the alveoli, in order to prevent that its weight to harm lung's structure, that was not developed to support large amounts of liquid inside. The weight, washing and whole body rotation will be controlled by the process computer.

Several optical cables, 1217, will be used for various purposes, as optical spectroscopy for various components fast, in-situ identification and concentration measurement, fluorescence spectroscopy and illumination. In some cases IR (infra-red) power may be introduced to warm up the alveolar wall or liquid, or a frequency selectively absorbed by a designed molecular compound present inside. A micro-imaging tubule, 1219, may carry a micro camera used as endoscope, and a measurement multi-micro-wire cable, 1218, used to further acquire more data during the lavage process, to assure success and controllability of the operation.

Optional, each cable may be equipped with a kind of myriapoda like advancement micro-motor, 1220, that may contain translational spikes, 1221, and rotational spikes, 1222, which assure cables better positioning inside and their stability, by anchoring themselves inside bronchiole.

Optional, cables may contain a radioactive tip made of different isotopes, and their position inside the body will be determinate from outside by a goniometer system with nuclear spectrometer, being important to correct the 3D images obtained by CT, MRI, or PET in agreement with patient's 3D position.

FIG. 13 depicts an adjustable position in 3D patient bed, as an embodiment of the present invention, with lavage system mechanics and fluidics incorporating many functional elements as power actuators and position control box, 1301, that have to be able to rotate about ½ ton mass, in a smooth fashion, exposing the alveolar walls in controlled manner, and with high accuracy.

As the system is hydro-pneumatic it requires cables and fluidics coming from power supply, 1302, to actuators, that are many, starting from a central arm turret, 1303, that is placed near center of mass, and contains a set of bearings with gear and actuators for two freedom degrees, 1304, that moves a telescopic inferior arm, 1305, which ends with an intermediary gear and actuator, 1306, which moves a secondary telescopic arm, 1307, which ends with a final gear and actuator—bed turret, 1308, with a two freedom degree articulation, 1309, that holds the bed structure, 1315, with everything that have to be on it.

As everything with patient is moving, the feed with necessary ingredients for lavage is made using respiratory gas tubes, 1310, placed near power box, as making the distances shorter is important, which are connected using feed pipes to lavage solution preparation, 1311, that drives the fluids to a plurality of mixer and pumping system, 1312, at least one for a lavage section. Return from lungs, coming from an exhaust pipe to the central hose, 1313, is connected to a central hose, carrying fluids, measurement tubes, 1314.

As bed and patient have to follow the liquid flow after a desired path in the alveoli, bed positioning system, 1316, have to be redundant and accurate in order to relate that to the room coordinate system and to patient lock in position cuffs, 1317, is such a manner that patient internal organs repositioning with the movement to be considered.

FIG. 14 gives a more detailed look at a system designed to hold the patient floating on the bed as immersed in a water pool, using pressure to discharge the weight, similar to an immersion in micro-gravity conditions, comprising a more complex hydro-pneumatic apparatuses, made to keep patient dry, while on pseudo buoyancy regime.

Patient, 1401, is embedded into hinged cylinders, with the head near the hose containing technologic tubules, 1402, connected to patient's bed structure, 1403.

Patient is positioned on a pseudo-water bed made of a half cylinder with a half cuff fixed on the bed, 1404, operating as a water bed supporting the patient all along, and covered by two quarter cylinders, 1405, on lateral hinges that are surrounding the patient, making the flotation feeling, split over torso and abdomen also varying pressure for helping the patient breathing.

When intending to work with hypo or hyper-bar regimes, as the human body is visco-plastic containing moving fluids inside, the pressure has to be equal all over within 1 Pa accuracy.

A helmet structure, 1406, holds the head in position, while a mouth piece, 1407, is passing technologic hose through helmet half cylinder covering the face, and is accommodating the hoses that are inserted in the mouth and cable passage for instruments, being water and air tight in the same time. It also have to be easy to be removed In case of emergency, or normal routine without inflicting any damage or discomfort for the patient. Vacuum may be applied to cuffs and outer tube, to make it shrink and be easily removed or introduced.

A thoracic tube and pressurized thoracic and abdominal cuffs, 1408, works together with an abdominal compression tube, 1409, to perform augmented breathing. The patient's body has to be surrounded by liquid, to provide buoyancy effect, and hip compression quarter cylinders, 1410, are placed in extension of the abdominal ones, and quarter cylinders covering basin legs, 1411, and arms for pressure equalizing are used to have all body coverage with the right pressure. Leg safety brackets, 1412, are used inside for setting patient secure in place, that are applied on hip too as hip safety brackets, 1414, for setting patient secure in place and arm safety brackets, 1415, are contributing to setting patient secure in place when the bed is rotating.

The bed movement is assured by a bed turret and gear actuator, 1416, connected to an upper arm with telescopic capability, 1417, to a middle joint, actuator and gearbox, 1418, and then to a lower telescopic arm, 1419, moved by a lower turret gearbox actuator and lower arm joint, 1421, placed over a power and hydraulics box, 1420, that hosts all actuator drivers and power boxes, 1422, assuring the smooth operation of the whole system.

The respiratory and lavage solutions are prepared using gas and liquids tubes, 1423, that are connected via connection fittings, 1425, to lavage and respiratory fluid preparation and gas preparation units, 1424, placed near the tanks.

Quality is assured by accurate measurements, and a flowmeter, volumeter, thermometer, manometer measuring unit, 1426, measuring continuously.

A plurality of tubes carrying lavage liquids, 1427, is joining in a common bunch to go up near articulated bed arm, to the helmet entry, and exhaust pipes going to liquid collector tanks that assures the fluidic good operation.

A plurality of lavage fluid drain tanks, 1428, are used for waste fluids recovery, and a good partitioning is needed for post process patient diagnosis, because these expectorates, with liquid together with a measurement system made of a plurality of measurement instrumentation and control valves, 1429, comprising a flow meter, volume meter, thermometer, manometer, sampler for laboratory analysis, optical spectrometry, and a sampling and measurement unit, contribute to the success of the operation and patient's long range treatment.

A control system, 1430, is synchronized with patient breathing helping patient breathe, is also measuring pressures, temperatures, flow, volume, composition, etc., and has an exhaust gas analyzer, that measures oxygen concentration, carbon dioxide, other gases, and transmits all data via a computer connection cable and data bus, 1431, for process control computer, 1432, connected to data acquisition units, that also acquires data from vibration, ultrasound and X Ray processing unit, 1433, performing complex calculations, mapping lung's efficiency by lobes, and making further predictions on patient's evolution in real time as controlling the good operation of lavage process. It is desired that an old lavaged patient to get younger lungs after the process being successfully performed, eliminating the damage from previous exposures.

FIG. 15 shows a connection view of process control system with specialized data acquisition, and data integration with visualization, that is able to access and integrate previous examinations results having a computer tomography (CT) compatible data port and converters, 1501, a Positron Emission Tomography (PET) compatible data port and converters, 1502, and a Nuclear Magnetic Resonance (MRI) compatible data port and converters, 1503, adapting data for display unit.

A system to visualize inside lungs for stereoscopic X ray imaging, 1504, comprises a pair of X-Ray generator tubes, 1505, mounted on a Gantry arm, 1506, rotating around patient, for stereoscopic and CT modes, generating images augmenting the previous examination results.

It is known that ultrasound propagates well in water, liquids and solids, even body tissue, but is strongly attenuated in the lung, except in this case of lavage where we adding water in the lung, and that allows a visualization inside the near pleura, up to 1″ above, therefore the usage of a plurality of Ultrasound Phased Array (USPA), 1507, for imaging, connected to an USPA image processing box, 1508, is highly recommended in order to indirectly detect the amount of water introduced in the lung, complementary to gamma ray attenuation measurement and imaging made using low radioactivity gamma sources placed on tubules tips.

Complementary, hydrophone receivers, 1509, connected to a hydrophone data processing box, 1510, for sound generator localization and sound partitioning, will detect and analyze any sound produced inside lungs, during breathing and lavage process, making the operators aware where are the chocking points in the wind channels.

Using liquid gravitational flow, is an enhancement compared with actual technology, but still not enough for a good comprehensive washing of the alveolar walls, therefore a plurality of Electromagnetic Hydro-tweeter (EHT), 1511, for vibration waves generation, connected to art EHT control unit, 1512, for phased array controlled washing pressure wave generation, that will be able to generate a moving wave in a lung's lobe fulfilled with lavage liquid, 1514.

Process control is performed by computer unit, 1515, containing a control panel and communication unit, 1516, a menu board on display unit, 1517, and is working networked with a lung immersion imaging unit, 1518, with a WiFi transmitter, 1519 to a WiFi receiver, 1521, of an Augmented Reality (AR) display, 1522, placed on the head of a mobile operator, 1520, acting together in the same room with a supervisor, 1523 that monitors the entire process.

FIG. 16—details a system to measure and analyze patient bio-parameters, and integrate in a process control system that has a data acquisition system, 1601, with computing simulation and visualization capabilities, as part of distributed computing power and redundancy by design, where a set or more, of wearable electronics, 1602, placed in all compartments that are holding the patient and are measuring bio-medical parameters are connected.

There are many bio-parameters to measure, with distributed sensors that may be customized as a function of the complexity of the operations but among those most common and important are, temperatures measurement, 1603, in various locations, 1604, pulse rate measurement, oxygen concentration in blood measurement, 1605, using simple sensors or combined sensor for oxygen concentration, pulse rate and temperature measurement, 1606, blood flow measurement by Doppler ultrasound, in exposed arteries as neck, arm, leg, 1607, if clogs are suspected as probably to form, due to anesthesia, breathing air gas concentration measurement, 1608, and other multiple pressure measurement sensors, 1609, for blood pressure on arms, and air pressure, other pressures in cuffs in real time, all ending up in a large data acquisition architecture.

A system to measure inside lung parameters, 1610, at the bronchiole level that may comprise many additional measurement and imaging devices, like a video camera, with optic fiber illumination system, 1611, acting as an endoscope, an optic fiber spectrometer, 1612, a manometer, 1613, for bronchiole pressure, gas analyzers, 1614, for each lavage tube ramification and breathing tube, very important to continuously measure the improvement in alveolar membrane gas exchange efficiency, and benchmarking the success of the operation, lavage temperature measurement, 1615, lavage liquid conductivity measurement, 1616, lavage liquids pH measurement, 1617, laser for fluorescence spectroscopy measurement, 1618, all these modules connected via a data bus, 1619, large band connection to computer.

For heating or sterilizing inside, a power IR laser, 1620, or any other tunable laser in vis and UV may be connected to fiber optic embedded in technologic lavage hose, 1621, in order to illuminate alveoli walls, 1622, in a consistent mode with prescribed lavage treatment.

A data fusion computer system, 1623, is used for integrating all information, coming via communication interfaces, 1624, with other process computers, for process visualization and control with quality assurance module, 1625, emergency procedures control with activation of emergency routines module, 1626, which may activate an automated defibrillator, 1627, and trigger emergency communication module, 1628, to make all necessary connections.

The entire concept is to monitor and interpret as many parameters as necessary to predict preemptive actions.

### EXAMPLES OF THE INVENTION

The current idea was to adapt a 3D flight simulator cockpit, to an operational room, where to use the degrees of mobility of the simulator, cockpit, now transformed into a bed infrastructure, that holds the patient in buoyancy in water, contained in pressurized bags, in any position needed for the liquid to fully fulfil an alveolar sac or a lung lobe, in and out carrying with it all residues, a surfactant solution might remove from the alveolar walls.

The present patent is not intended to drastically change how the lavage is made, it does not bring new solutions in spite it highlights the need for a more sustained research in this direction, but offers the possibility of applying a sequence of washing, rinsing, healing and improving, placing the patient in any desired position, for optimal process. It also allows a large variety of pressures from −0.5 bar up to 2-3 bar as deemed optimal for the medical treatment.

When the present invention is applied to a patient, with lung damage, first in the absence of a CT, a stereoscopic X ray visualization is performed from various angles to best identify the lobes first to treat and lobes used for blood oxygenation. Then the patient is placed under anesthesia, and tubes are introduced through the mouth in the lungs, down to tertiary trachea and air tight there, while the liquids are tighten around the body, and air pressure is applied to assist breathing. The bed with the patient is rotated in the desired position, prescribed lavage liquid is pouring in the alveolar sac, fulfilling it, and from outside a vibration is applied to agitate the liquid inside alveoli, to better wash the mucus and residues there. Taking advantage that that lung area is full with liquid ultrasound visualization is used for inside imaging.

The procedure starts with the patient in a position aligning the lung's lobe bronchiole with gravitational acceleration, such as the lavage liquid to fall freely on the bottom of the alveoli, and fulfill them up to top, in the bronchiole plug, where during this operation air is gradually removed. Than ultrasound visualization, and vibration shake-up the liquid is applied in order to improve the washing factor. Simultaneously the hydrophones are used to detect if any other sounds are produced, due to collateral liquid leakage in damaged lung during breathing.

After few cycles the patient is flipped in the opposite position with the cuff and leakage rubes down, such as the lavage liquid to drain naturally, while air is added and vibration is maintained to remove all solid, viscous and fluid effluents. The input fluid and output fluid volumes are measured. Other chemical and bio-medical measurements may be performed on output fluid, with positional assignation of the findings, as to map the lungs.

Process is repeated several times, until the entire lobe zone restoration procedure is completed.

Finally the lung zone efficiency is measured by measuring the gas parameters before and after respiration. This is important information to control the near-by lobes operation, because

During the lavage of one lobe partition the rest of the lung is oxygenated, and used for patient's breathing. The pneumatic cuff around the hydraulic cuff is helping breathing pressurizing and depressurizing over the abdomen and ribs to assist the muscles.

The hydraulic cuff may create pressure or negative pressure in agreement with outside breathing gas and lavage liquid supply, in order to apply the treatment at the most effective pressure. Blood pressure and health of the blood transport system have to be analyzed when negative pressures are used, to prevent any blood spill accident.

When successful, this technology may reduce the thickness time and improve life expectancy, by bringing back to initial values or better the lung operating parameters.

1) A system to improve lung lavage operation comprising:
a.—Adjustable position in 3D patient bed, made of:
I—power and control box;
II—articulated arms with gears and actuator boxes;
III—fluidic reservoirs and mixers;
IV—Bed positioning system;
V—Patient lock in position cuffs;
b)—System to hold the patient floating on the bed comprising:
I—A set of hydro-pneumatic cuffs, working together air tight to control pressure, applied to patient, embedded into hinged cylinders, where each cuff is including:
*—A half cylinder with a half cuff fixed on the bed, operating as a water bed supporting the patient all along;
**—Two quarter cylinders on lateral hinges that are surrounding the patient, making the flotation feeling, split over torso and abdomen also varying pressure for helping the patient breathing;
***—Quarter cylinders covering basin legs, and arms for pressure equalizing;
****—Helmet half cylinder covering the face, and accommodating the hoses that are inserted in the mouth;
II—Safety brackets for setting patient secure in place;
III—Cable passage for instruments, being water and air tight;
c)—A plurality of fluidic systems to lavage the patient, comprising:
I—A set of gas and lavage liquids tanks, placed adjacent to power box;
II—A plurality of gas preparation units placed near the tanks;
III—A plurality of tubes carrying lavage liquids, joining in a common bunch to go up near articulated bed arm, to the helmet entry;
IV—A plurality of lavage fluid drain tanks, for waste fluids recovery;
V—A plurality of measurement instrumentation and control valves comprising:
A. Flow meter;
B. Volume meter;
C. Temperature;
D. Pressure;
E. Sampler for laboratory analysis;
F. Optical spectrometry;
d)—A system to assist patient breathing, made of:
I—Respirator gas preparation unit;
II—Control system synchronized with patient;
III—Pressurized thoracic and abdominal cuffs;
IV—Exhaust gas analyzer;
V—Laboratory analyses sampler;
e)—A system to visualize inside lungs, comprising:
I—CT, PET, MRI compatible display unit;
II—Stereoscopic X ray visualization device;
III—A plurality of Ultrasound phased arrays;
IV—A plurality of hydrophones inside hydraulic cuffs, locating the sound sources inside the lung;
f)—A system to vibrate the alveoli made of:
I—A plurality of underwater tweeter loudspeakers, working as phased arrays;
II—A ultrasound imaging and location systems that controls the aiming;
g)—A system to measure and analyze patient bio-parameters, comprising:
I—A data acquisition system with computing simulation and visualization capabilities;
II—A set of wearable electronics placed in all compartments holding the patient, measuring temperatures, pulse rate, oxygen, pressures, blood flow, etc.;
III—A system to measure inside lung parameters at the bronchiole level that may comprise:
A)—Video camera, illumination system;
B)—Optic fiber spectrometer;
C)—Bronchiole pressure;
D)—Gas analyzer at sub-lobe level;
E)—Temperature;
F)—Conductivity and pH;
IV—A optic fiber to apply optic power to selectively worm up or excite tissue;
h)—A system to control process, comprising:
I.—A computer system integrating all information;
II.—Communication interfaces with other process computers;
III.—Process visualization and control with quality assurance;
IV.—Emergency procedures control with activation of:
A)—Defibrillator;
B)—Release patient procedure;
C)—Emergency communication.

2) A system to improve lung lavage operation according claim 1, where the patient is hydraulically supported like on his buoyancy and rotated in all positions to guide naturally fluid movement inside the desired lung lobe.

3) A system to improve lung lavage operation according claim 1, where the patient may be treated in hypobaric conditions, to increase expectoration, or hyperbaric conditions to increase the oxygen exchange or nitrogen absorption to create favorable treatment conditions.

4) A system to improve lung lavage operation according claim 1, that monitors continuously all necessary bio-medical parameters for quality assurance purposes;

5) A system to improve lung lavage operation according claim 1, where lung-lobe loading with lavage liquid is monitored versus lung's elasticity in order to avoid any alveolar damage;

6) A system to improve lung lavage operation according claim 1, where radioactive goniometry, and gamma ray absorption scanning is used to profile the lung lavage liquid loading;

7) A system to improve lung lavage operation according claim 1, that uses pseudo-buoyancy to float the patient in contained liquids, and rotate gently in order to avoid any lung excessive stress;

8) A system to improve lung lavage operation according claim 1, where the lavage solution is a mixture, gas fluid, applied at various temperatures and pressure regimes;

9) A system to improve lung lavage operation according claim 1, where laser pushes power inside at various frequencies, to heat up solution or alveolar walls or to attack various molecular compounds inside;

10) A system to improve lung lavage operation according claim 1, where, the efficacy of lungs gas exchange is measured continuously based on gas analysis;

11) A method for lung lavage that has the following steps:
a)—Have the preliminary imagery and diagnosis transferred in process control computer;
b)—Elaborate lung treatment process plan;
c)—Bring patient and transfer in the lavage bed under anesthesia;
d)—Connect the bio-parameter measurement system;
e)—Close the hydro-pneumatic cuffs and lock patient in position;
f)—Introduce the process tube via mouth and trachea in the lungs;
g)—Make every tube advance using the bending and stepper/rotator mechanisms on tube;
h)—Monitor the advancement of tubes inside bronchiole using X-ray goniometry;
i)—Use the breathing tube to maintain normal respiration of patient;
j)—With the tubules in place make a X-ray stereoscopic radiography, in various positions to check the accuracy of tubule placement, digitize and overlap on CT, MRI, PET pre-existent data;
k)—Place each tubule in place and inflate cuffs sealing air tight the lung regions;
l)—Rotate the patient until the selected lung lobe for lavage have the alveoli placed in the right position for being filled with lavage prewash liquid;
m)—Start liquid filling, while assisting breathing, and monitoring bio-parameters (vitals included);
n)—Use ultrasound phased arrays to image the lung flooded zone, and vibration generator to make the liquid wash alveolar walls, and proceed for a prescribed time, while rotating patient to cover all alveolar surface and washing angles;
p)—Flip patient in the drainage position and gently drain liquid while introducing fulfillment gases for the first step;
q)—Flip back the patient and start next step, with the other set of prescribed liquids;
r)—Repeat points m, n, p;
s)—Repeat q, r as needed according to treatment procedure;
t)—When procedure completed, set the patient in initial position, and measure the lobe with had lavage functionality, than switch on the next lobe scheduled for lavage;
u)—Start the lavage procedure and repeat m-t;
v)—Bring patient in the initial position and reposition the hoses inside lungs, and repeat g-u;
x)—When finish, bring the patient in the initial position and measure image and make all end of procedure tests and quality assurance measurements;
y)—Bring all parameters to ambient pressure, measure and check again, withdraw the tubes from patient, and measure again the global functionality;
z)—Release the patient and transfer on the transport bed.

12) A method for lung lavage according claim 11, that may be customized on various lung clogging diseases, using various treatment procedures;

13) A method for lung lavage according claim 11, which may have four main phases of lavage, that are:
a) pre-clean and clean the lung lobe from mucus and other depositions;
b) kill the viruses and bacteria;
c) heal alveolar wall and bronchi;
d) rinse the lung's lobe and apply enhancers and measure the functionality;

14) A method for lung lavage according claim 11, where the patient may be rotated on any azimuthal and polar angle in order to allow a lavage liquid inserted in alveoli to wash and cover naturally all the walls and remove and train the depositions towards exhaust hole;

15) A method for lung lavage according claim 11 than uses pressures in both hyperbaric and hypobaric domain, in order to increase the effectiveness of the treatment;

16) A method for lung lavage according claim 11, that is measuring and mapping the lung's performances and bio-medical parameters during the entire procedure;

17) A method for lung lavage according claim 11, that uses vibration to increase the lung's lobes lavage efficiency;

18) A method for lung lavage according claim 11, that uses gamma ray goniometry and imaging in order to set the position of hoses inside lung with high accuracy;

19) A method for lung lavage according claim 11, that uses sound listening and localization in order to diagnose potential issues, of lung functionality;

20) A method for lung lavage according claim 11, that uses laser lung irradiation in order to kill bacteria and viruses during lavage procedure.
